(19) United States
(12) Patent Application Publication
(10) Pub. No.: US 2010/0063092 A1
(43) Pub. Date: Mar. 11, 2010

(54) 3-CYANO-4-(4-PHENYL-PIPERIDIN-1-YL)-PYRIDIN-2-ONE DERIVATIVES

(75) Inventors: José Maria Cid-Núñez, Toledo (ES); Andrés Avelino Trabanco-Suárez, Olias del Ray (ES); Gregor James MacDonald, Zoersel (GB); Guillaume Albert Jacques Duvey, Saint Julien en Genevois (FR); Robert Johannes Lütjens, Geneva (CH)

Correspondence Address: GARDNER GROFF GREENWALD & VILLANUEVA. PC, 2018 POWERS FERRY ROAD, SUITE 800, ATLANTA, GA, 30339, US

(73) Assignee: Ortho-Mcneil-Janssen Pharmaceuticals, Inc., Titusville, NJ (US)

(21) Appl. No.: 12/529,632

(22) PCT Filed: Mar. 7, 2008
(86) PCT No.: PCT/EP2008/052766

§ 371 Date: Sep. 2, 2009

(30) Foreign Application Priority Data
Mar. 7, 2007 (EP) 07103654.5
Sep. 14, 2007 (EP) 07116401.6

(51) Int. Cl. A61K 31/4545; C07D 401/04; A61P 25/00

(52) U.S. Cl. 514/318; 546/194

(57) ABSTRACT

The present invention relates to novel compounds, in particular novel pyridinone derivatives according to Formula (I) including any stereochemically isomeric form thereof, or a pharmaceutically acceptable salt thereof or a solvate thereof, wherein all radicals are defined in the application and claims. The compounds according to the invention are positive allosteric modulators of metabotropic glutamate receptors subtype 2 (“mGluR2”) which are useful for the treatment or prevention of neurological and psychiatric disorders associated with glutamate dysfunction and diseases in which the mGluR2 subtype of metabotropic receptors is involved. In particular, such diseases are central nervous system disorders selected from the group of anxiety, schizophrenia, migraine, depression, and epilepsy. The invention is also directed to pharmaceutical compositions and processes to prepare such compounds and such compositions, as well as to the use of such compounds for the prevention and treatment of such diseases in which mGluR2 is involved.

### FIELD OF THE INVENTION

The present invention relates to novel pyridin-2-one-derivatives which are positive allosteric modulators of the metabotropic glutamate receptor subtype 2 (“mGluR2”) and which are useful for the treatment or prevention of neurological and psychiatric disorders associated with glutamate dysfunction and diseases in which the mGluR2 subtype of metabotropic receptors is involved. The invention is also directed to pharmaceutical compositions comprising such compounds, to processes to prepare such compounds and such compositions, and to the use of such compounds for the prevention or treatment of neurological and psychiatric disorders and diseases in which mGluR2 is involved.

### BACKGROUND OF THE INVENTION

Glutamate is the major amino acid neurotransmitter in the mammalian central nervous system. Glutamate plays a major role in numerous physiological functions, such as learning and memory but also sensory perception, development of synaptic plasticity, motor control, respiration, and regulation of cardiovascular function. Furthermore, glutamate is at the centre of several different neurological and psychiatric diseases, where there is an imbalance in glutamatergic neurotransmission.

Glutamate mediates synaptic neurotransmission through the activation of ionotropic glutamate receptors channels (iGluRs), and the NMDA, AMPA and kainate receptors which are responsible for fast excitatory transmission.

In addition, glutamate activates metabotropic glutamate receptors (mGluRs) which have a more modulatory role that contributes to the fine-tuning of synaptic efficacy.

Glutamate activates the mGluRs through binding to the large extracellular amino-terminal domain of the receptor, herein called the orthosteric binding site. This binding induces a conformational change in the receptor which results in the activation of the G-protein and intracellular signalling pathways.

The mGluR2 subtype is negatively coupled to adenylate cyclase via activation of Gαi-protein, and its activation leads to inhibition of glutamate release in the synapse. In the central nervous system (CNS), mGluR2 receptors are abundant mainly throughout cortex, thalamic regions, accessory olfactory bulb, hippocampus, amygdala, caudate-putamen and nucleus accumbens.

Activating mGluR2 was shown in clinical trials to be efficacious to treat anxiety disorders. In addition, activating mGluR2 in various animal models was shown to be efficacious, thus representing a potential novel therapeutic approach for the treatment of schizophrenia, epilepsy, addiction/drug dependence, Parkinson's disease, pain, sleep disorders and Huntington's disease.

To date, most of the available pharmacological tools targeting mGluRs are orthosteric ligands which activate several members of the family as they are structural analogs of glutamate.

A new avenue for developing selective compounds acting at mGluRs is to identify compounds that act through allosteric mechanisms, modulating the receptor by binding to a site different from the highly conserved orthosteric binding site.

Positive allosteric modulators of mGluRs have emerged recently as novel pharmacological entities offering this attractive alternative. Various compounds have been described as mGluR2 positive allosteric modulators.

WO2004/092135 (NPS & Astra Zeneca), WO2004/018386, WO2006/014918 and WO2006/015158 (Merck), WO2001/56990 (Eli Lilly) and WO2006/030032 (Addex & Janssen Pharmaceutica) describe respectively phenyl sulfonamide, acetophenone, indanone, pyridylmethyl sulfonamide and pyridinone derivatives as mGluR2 positive allosteric modulators. None of the specifically disclosed compounds are structurally related to the compounds of the invention.

WO2007/104783 describes 1,4-disubstituted 3-cyano-pyridone-derivatives that are positive allosteric modulators of metabotropic receptors-subtype 2 (“mGluR2”).

It was demonstrated that such compounds do not activate the receptor by themselves. Rather, they enable the receptor to produce a maximal response to a concentration of glutamate which by itself induces a minimal response. Mutational analysis has demonstrated unequivocally that the binding of mGluR2 positive allosteric modulators does not occur at the orthosteric site, but instead at an allosteric site situated within the seven transmembrane region of the receptor.

Animal data are suggesting that positive allosteric modulators of mGluR2 have effects in anxiety and psychosis models similar to those obtained with orthosteric agonists. Allosteric modulators of mGluR2 were shown to be active in fear-potentiated startle, and in stress-induced hyperthermia models of anxiety. Furthermore, such compounds were shown to be active in reversal of ketamine- or amphetamine-induced hyperlocomotion, and in reversal of amphetamine-induced disruption of prepulse inhibition of the acoustic startle effect models of schizophrenia (J. Pharmacol. Exp. Ther. 2006, 318, 173-185; Psychopharmacology 2005, 179, 271-283).

Recent animal studies further reveal that the selective positive allostric modulator of metabotropic glutamate receptor subtype 2 biphenyl-indanone (BINA) blocks a hallucinogenic drug model of psychosis, supporting the strategy of targeting mGluR2 receptors for treating glutamatergic dysfunction in schizophrenia (Mol. Pharmacol. 2007, 72, 477-484).

Positive allosteric modulators enable potentiation of the glutamate response, but they have also been shown to potentiate the response to orthosteric mGluR2 agonists such as LY379268 or DCG-IV. These data provide evidence for yet another novel therapeutic approach to treat above mentioned neurological and psychiatric diseases involving mGluR2, which would use a combination of a positive allosteric modulator of mGluR2 together with an orthosteric agonist of mGluR2.

The present compounds are characterized by a central pyridine-2-one moiety substituted in position 3 with cyano and in position 4 with piperidin-1-yl which is in turn substituted with phenyl in position 4. The present compounds are potent positive allosteric mGluR2 modulators.

### DESCRIPTION OF THE INVENTION

The invention relates to compounds having metabotropic glutamate receptor 2 modulator activity. The present invention provides a compound according to formula (I),

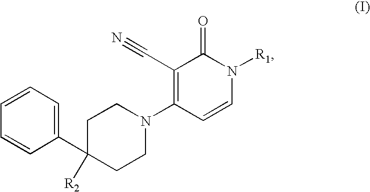

including any stereochemically isomeric form thereof, wherein
R₁ is C₄₋₆alkyl, or C₁₋₃alkyl substituted with C₃₋₇cycloalkyl;
R₂ is hydrogen; fluoro; C₁₋₄alkyl substituted with hydroxyl; C₁₋₄alkyl substituted with fluoro; or C₁₋₄alkyloxy substituted with fluoro;
or a pharmaceutically acceptable salt thereof or a solvate thereof.

The present invention also relates to the use of a compound of formula (I) or any subgroup thereof for the manufacture of a medicament for treating or preventing, in particular for treating, a condition in a mammal, including a human, the treatment or prevention of which is affected or facilitated by the neuromodulatory effect of an allosteric modulator of mGluR2, in particular a positive allosteric modulator.

An embodiment of the present invention are those compounds of formula (I),

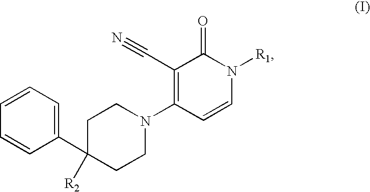

including any stereochemically isomeric form thereof, wherein
R₁ is C₄₋₆ alkyl, or C₁₋₃alkyl substituted with C₃₋₇cycloalkyl;
R₂ is hydrogen; fluoro; C₁₋₄ alkyl substituted with hydroxyl; C₁₋₄alkyl substituted with fluoro; or C₁₋₄alkyloxy substituted with fluoro;
or a pharmaceutically acceptable salt thereof or a solvate thereof;
provided that the compound is other than

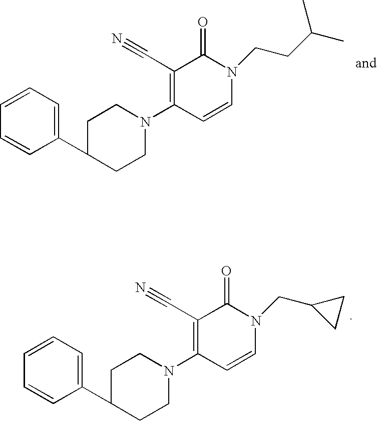

An embodiment of the present invention are those compounds of formula (I) wherein R₁ is C₄₋₆ alkyl, in particular C₄₋₅ alkyl, such as for example 1-butyl, 2-methyl-1-propyl, 3-methyl-1-butyl; in particular 1-butyl.

An embodiment of the present invention are those compounds of formula (I) wherein R₁ is C₁₋₃ alkyl substituted with C₃₋₇ cycloalkyl, in particular cyclopropylmethyl or 2-(cyclopropyl)-1-ethyl.

An embodiment of the present invention are those compounds of formula (I) or any subgroup thereof as mentioned hereinbefore as embodiment, wherein R₂ is hydrogen.

An embodiment of the present invention are those compounds of formula (I) or, whenever possible, any subgroup thereof as mentioned hereinbefore as embodiment, wherein R₂ is fluoro; C₁₋₄alkyl substituted with hydroxyl; C₁₋₄ alkyl substituted with fluoro; or C₁₋₄alkyloxy substituted with fluoro.

An embodiment of the present invention are those compounds of formula (I) or, whenever possible, any subgroup thereof as mentioned hereinbefore as embodiment, wherein R₂ is hydrogen; fluoro; or C₁₋₄ alkyloxy substituted with fluoro; in particular R₂ is fluoro or C₁₋₄alkyloxy substituted with fluoro.

An embodiment of the present invention are those compounds of formula (I) or any subgroup thereof as mentioned hereinbefore as embodiment, wherein R₂ is fluoro.

An embodiment of the present invention are those compounds of formula (I) or, whenever possible, any subgroup thereof as mentioned hereinbefore as embodiment, wherein R₂ is C₁₋₄alkyl substituted with hydroxyl, in particular wherein R₂ is methyl substituted with hydroxyl.

An embodiment of the present invention are those compounds of formula (I) or, whenever possible, any subgroup thereof as mentioned hereinbefore as embodiment, wherein R₂ is C₁₋₄ alkyl substituted with fluoro, in particular wherein R₂ is methyl substituted with fluoro.

An embodiment of the present invention are those compounds of formula (I) or, whenever possible, any subgroup thereof as mentioned hereinbefore as embodiment, wherein R₂ is C₁₋₄ alkyloxy substituted with fluoro, in particular wherein R₂ is ethyloxy substituted with fluoro.

An embodiment of the present invention are those compounds of formula (I), wherein R₁ is C₄₋₆ alkyl, in particular 1-butyl or 3-methyl-1-butyl; or C₁₋₃ alkyl substituted with C₃₋₇ cycloalkyl, in particular cyclopropylmethyl;

R₂ is hydrogen; fluoro; C₁₋₄alkyl substituted with hydroxyl, in particular methyl substituted with hydroxyl; C₁₋₄ alkyl substituted with fluoro, in particular methyl substituted with fluoro; or C₁₋₄alkyloxy substituted with fluoro, in particular ethyloxy substituted with fluoro;
or a pharmaceutically acceptable salt thereof or a solvate thereof.

An embodiment of the present invention are those compounds of formula (I), wherein R₁ is C₄₋₆ alkyl, in particular 1-butyl; or C₁₋₃ alkyl substituted with C₃₋₇ cycloalkyl, in particular cyclopropylmethyl;

R₂ is fluoro; C₁₋₄alkyl substituted with hydroxyl, in particular methyl substituted with hydroxyl; or C₁₋₄alkyl substituted with fluoro, in particular methyl substituted with fluoro; or C₁₋₄alkyloxy substituted with fluoro, in particular ethyloxy substituted with fluoro;
or a pharmaceutically acceptable salt thereof or a solvate thereof.

An embodiment of the present invention are those compounds of formula (I) selected from,

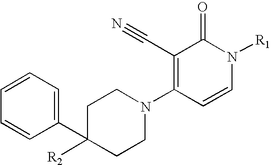

| 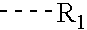 | 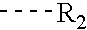 |
|---|---|
| 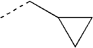 | 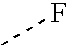 |
| 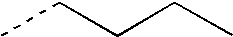 | 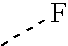 |
| 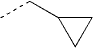 | 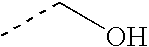 |
| 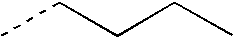 | 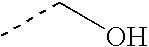 |
| 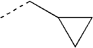 | 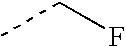 |
| 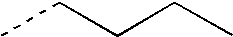 | 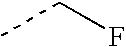 |
| 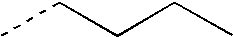 | 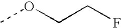 |
| 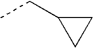 | H |
| 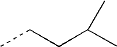 | H |

or a pharmaceutically acceptable salt thereof or a solvate thereof.

An embodiment of the present invention are those compounds of formula (I) selected from,

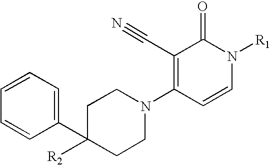

| 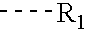 | 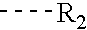 |
|---|---|
| 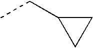 | 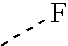 |
| 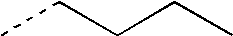 | 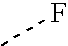 |
| 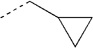 | 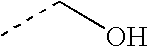 |
| 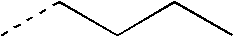 | 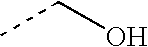 |
| 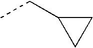 | 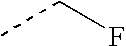 |
| 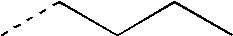 | 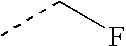 |
| 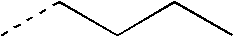 | 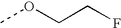 |

or a pharmaceutically acceptable salt thereof or a solvate thereof.

As used hereinbefore or hereinafter, the notation C₁₋₃ alkyl as a group or part of a group defines a saturated, straight or branched, hydrocarbon radical having from 1 to 3 carbon atoms, such as methyl, ethyl, 1-propyl and 1-methyl-1-ethyl.

As used hereinbefore or hereinafter, the notation C₁₋₄alkyl as a group or part of a group defines a saturated, straight or branched, hydrocarbon radical having from 1 to 4 carbon atoms such as methyl, ethyl, propyl, 1-methyl-1-ethyl, 1-butyl, 2-methyl-1-propyl. Preferably, C₁₋₄alkyl represents methyl.

As used hereinbefore or hereinafter, the notation C₄₋₆alkyl as a group or part of a group defines a saturated, straight or branched, hydrocarbon radical having from 4 to 6 carbon atoms such as 1-butyl, 2-methyl-1-propyl, 1-pentyl, 2-methyl-1-butyl, 3-methyl-1-butyl, 1-hexyl and the like.

As used hereinbefore or hereinafter, the notation C₄₋₅alkyl as a group or part of a group defines a saturated, straight or branched, hydrocarbon radical having 4 or 5 carbon atoms such as 1-butyl, 2-methyl-1-propyl, 1-pentyl, 2-methyl-1-butyl, 3-methyl-1-butyl and the like.

As used hereinbefore or hereinafter, the notation C₃₋₇ cycloalkyl defines a saturated, cyclic hydrocarbon radical having from 3 to 7 carbon atoms, such as cyclopropyl, cyclobutyl, cyclopentyl, cyclohexyl and cycloheptyl. Preferably C₃₋₇cycloalkyl represents cyclopropyl.

For therapeutic use, salts of the compounds of formula (I) are those wherein the counterion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are included within the ambit of the present invention.

The pharmaceutically acceptable salts are defined to comprise the therapeutically active non-toxic acid addition salt forms that the compounds according to formula (I) are able to form. Said salts can be obtained by treating the base form of the compounds according to formula (I) with appropriate acids, for example inorganic acids, for example hydrohalic acid, in particular hydrochloric acid, hydrobromic acid, sulphuric acid, nitric acid and phosphoric acid; organic acids, for example acetic acid, hydroxyacetic acid, propanoic acid, lactic acid, pyruvic acid, oxalic acid, malonic acid, succinic acid, maleic acid, fumaric acid, malic acid, tartaric acid, citric acid, methanesulfonic acid, ethanesulfonic acid, benzenesulfonic acid, p-toluenesulfonic acid, cyclamic acid, salicylic acid, p-aminosalicylic acid and pamoic acid.

Conversely said acid salt forms can be converted into the free base form by treatment with an appropriate base.

The compounds according to formula (I) containing acidic protons may also be converted into their therapeutically active non-toxic base salt forms by treatment with appropriate organic and inorganic bases. Appropriate base salt forms comprise, for example, the ammonium salts, the alkaline and earth alkaline metal salts, in particular lithium, sodium, potassium, magnesium and calcium salts, salts with organic bases, e.g. the benzathine, N-methyl-D-glucamine, hybramine salts, and salts with amino acids, for example arginine and lysine.

Conversely, said base salt forms can be converted into the free acid forms by treatment with an appropriate acid.

The pharmaceutically acceptable acid addition salt forms of the compounds of formula (I) are the preferred pharmaceutically acceptable salt forms of the compounds of formula (I).

The term solvate comprises the solvent addition forms as well as the pharmaceutically acceptable salts thereof, which the compounds of formula (I) are able to form. Examples of such solvent addition forms are e.g. hydrates, alcoholates and the like.

It will be appreciated that some of the compounds of formula (I) and their salts and solvates may contain one or more centers of chirality and exist as stereochemically isomeric forms.

The term “stereochemically isomeric forms” as used hereinbefore defines all the possible isomeric forms that the compounds of formula (I) may possess. Unless otherwise mentioned or indicated, the chemical designation of compounds denotes the mixture of all possible stereochemically isomeric forms, said mixtures containing all diastereomers and enantiomers of the basic molecular structure. But the invention also embraces each of the individual isomeric forms of formula (I) and their salts or solvates, substantially free, i.e. associated with less than 10%, preferably less than 5%, in particular less than 2% and most preferably less than 1% of the other isomers. Thus, when a compound of formula (I) is for instance specified as (R), this means that the compound is substantially free of the (S) isomer.

In particular, stereogenic centers may have the R- or S-configuration; substituents on bivalent cyclic (partially) saturated radicals may have either the cis- or trans-configuration.

Following CAS nomenclature conventions, when two stereogenic centers of known absolute configuration are present in a compound, an R or S descriptor is assigned (based on Cahn-Ingold-Prelog sequence rule) to the lowest-numbered chiral center, the reference center. The configuration of the second stereogenic center is indicated using relative descriptors [R*,R*] or [R*,S*], where R* is always specified as the reference center and [R*,R*] indicates centers with the same chirality and [R*,S*] indicates centers of unlike chirality. For example, if the lowest-numbered chiral center in the compound has an S configuration and the second center is R, the stereo descriptor would be specified as S—[R*,S*]. If “α” and “β” are used: the position of the highest priority substituent on the asymmetric carbon atom in the ring system having the lowest ring number, is arbitrarily always in the “α” position of the mean plane determined by the ring system. The position of the highest priority substituent on the other asymmetric carbon atom in the ring system (hydrogen atom in compounds according to Formula (I)) relative to the position of the highest priority substituent on the reference atom is denominated “α”, if it is on the same side of the mean plane determined by the ring system, or “β”, if it is on the other side of the mean plane determined by the ring system.

Whenever used hereinafter, the term “compounds of formula (I)” or any subgroup thereof, is meant to also include their stereochemically isomeric forms, their pharmaceutically acceptable salts and their solvates. Of special interest are those compounds of formula (I) which are stereochemically pure.

In the framework of this application, an element, in particular when mentioned in relation to a compound according to formula (I), comprises all isotopes and isotopic mixtures of this element, either naturally occurring or synthetically produced, either with natural abundance or in an isotopically enriched form. In particular, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluor is mentioned, it is understood to refer to ¹⁸F, ¹⁹F or mixtures thereof. The compounds according to the invention therefore also comprise compounds with one or more isotopes of one or more element, and mixtures thereof, including radioactive compounds, also called radio labelled compounds, wherein one or more non-radioactive atoms has been replaced by one of its radioactive isotopes. In particular, the radioactive atom is selected from the group of hydrogen, carbon, nitrogen, sulfur, oxygen and halogen. Preferably, the radioactive atom is selected from the group of hydrogen, carbon and halogen. In particular, the radioactive isotope is selected from the group of ³H, ¹¹C, ¹⁸F, ¹²²I, ¹²³ I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

Whenever used hereinbefore or hereinafter that substituents can be selected each independently out of a list of definitions, all possible combinations are intended which are chemically possible.

In general, compounds of formula (I) can be prepared according to the below Experimental procedures 1 to 4.

### Experimental Procedure 1

The compounds according to formula (I) can be prepared by reacting an intermediate of formula (II) wherein Y represents a suitable leaving group, such as for example F₃C—S(═O)₂—O— or halo, e.g. bromo and the like, with an intermediate of formula (III) according to Reaction Scheme (1). The reaction can be performed in a suitable reaction-inert solvent, such as, for example, dimethoxyethane or acetonitrile, in the presence of a suitable base, such as, for example, Cs₂CO₃ or N,N-diisopropylethylamine, under thermal conditions such as, for example, heating the reaction mixture for example at 150° C. under microwave irradiation for 15 minutes.

The reaction described in Reaction Scheme (1) can also be performed in a suitable reaction-inert solvent, such as, for example, 1,4-dioxane, in the presence of a suitable base, such as, for example, K₃PO₄, a suitable catalyst, such as a Pd-complex catalyst such as, for example,

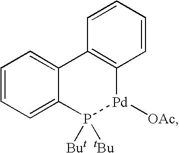

under thermal conditions such as, for example, heating the reaction mixture for example at 80° C. for 12 hours.
In reaction scheme (1), all variables are defined as in formula (I).

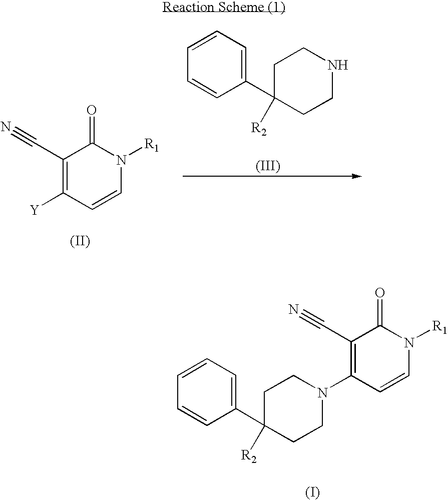

### Experimental Procedure 2

Compounds of formula (I) wherein R₂ represents C₁₋₄ alkyl substituted with fluoro, said C₁₋₄ alkyl being represented by L and said compounds being represented by formula (I-a), can be prepared by reacting a compound of formula (I) wherein R₂ represents C₁₋₄ alkyl substituted with hydroxyl, said compound being represented by formula (I-b), with a suitable fluorinating agent such as for example (diethylamino)sulfur trifluoride [CAS: 38078-09-0]. The reaction can be performed in a suitable reaction-inert solvent, such as for example dichloromethane, under a moderately low temperature such as, for example, a temperature ranging from −78° C. to 30° C. during for example 0.5 to 12 hours.

In Reaction Scheme (2), all variables are defined as in formula (I).

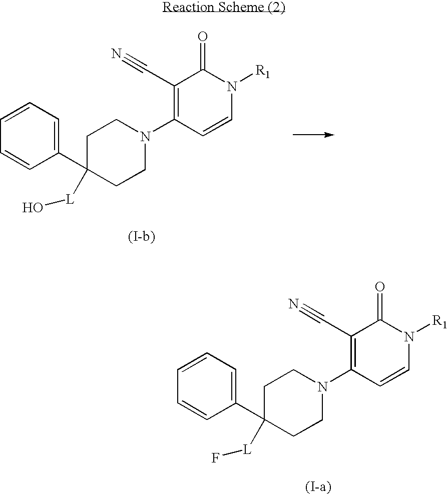

### Experimental Procedure 3

Compounds of formula (I) wherein R₂ represents fluoro, said compounds being represented by formula (I-c), can be prepared by reacting an intermediate of formula (IV) with a suitable fluorinating agent such as for example (diethylamino)sulfur trifluoride. The reaction can be performed in a suitable reaction-inert solvent, such as for example dichloromethane, under a moderately low temperature such as, for example, a temperature ranging from −78° C. to 30° C. during for example 0.5 to 12 hours.

In Reaction Scheme (3), all variables are defined as in formula (I).

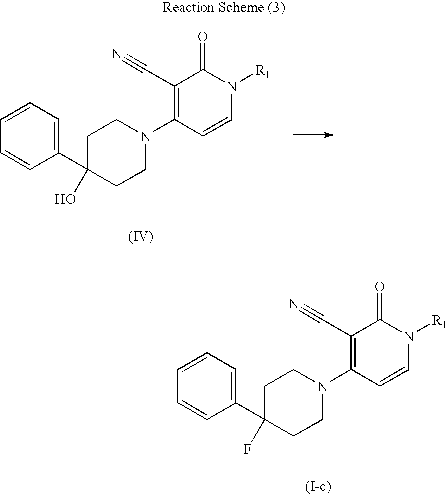

### Experimental Procedure 4

Compounds of formula (I) wherein R₂ represents C₁₋₄alkyloxy substituted with fluoro, said R₂ being represented by R′₂ and said compounds being represented by formula (I-d), can be prepared by hydrogenating an intermediate of formula (XIV), according to Reaction Scheme (4). The reaction can be performed in a suitable solvent, such as for example an alcohol, e.g. methanol, in the presence of a suitable catalyst, such as for example palladium on activated carbon, and a suitable base, such as for example triethylamine. In reaction scheme (4), all variables are defined as in formula (I).

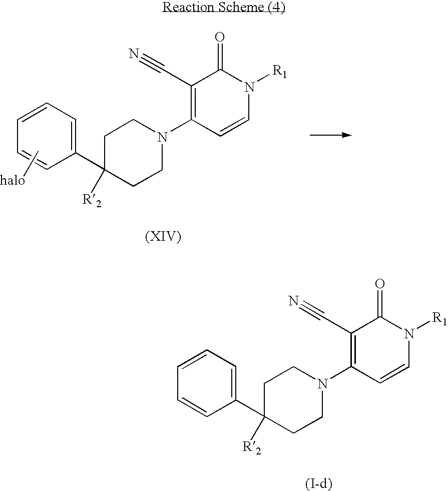

The compounds of formula (I) and some of the intermediates in the present invention may contain an asymmetric carbon atom. Pure stereochemically isomeric forms of said compounds and said intermediates can be obtained by the application of art-known procedures. For example, diastereoisomers can be separated by physical methods such as selective crystallization or chromatographic techniques, e.g. counter current distribution, chiral liquid chromatography and the like methods. Enantiomers can be obtained from racemic mixtures by first converting said racemic mixtures with suitable resolving agents such as, for example, chiral acids, to mixtures of diastereomeric salts or compounds; then physically separating said mixtures of diastereomeric salts or compounds by, for example, selective crystallization or chromatographic techniques, e.g. liquid chromatography and the like methods; and finally converting said separated diastereomeric salts or compounds into the corresponding enantiomers. Pure stereochemically isomeric forms may also be obtained from the pure stereochemically isomeric forms of the appropriate intermediates and starting materials, provided that the intervening reactions occur stereospecifically.

An alternative manner of separating the enantiomeric forms of the compounds of formula (I) and intermediates involves liquid chromatography or SCF (Super Critical Fluid) chromatography, in particular using a chiral stationary phase.

Some of the intermediates and starting materials are known compounds and may be commercially available or may be prepared according to art-known procedures.

The intermediates can also be prepared according to the below Experimental procedures 5 to 13.

### Experimental Procedure 5

Intermediates of formula (II) wherein Y represents halo, said intermediates being represented by formula (II-a), can be prepared by reacting an intermediate of formula (V) with a suitable halogenating agent such as, for example, P(═O)Br₃ according to Reaction Scheme (5). The reaction can be performed in a suitable reaction-inert solvent such as, for example, DMF, at a moderately elevated temperature such as, for example, 110° C. In Reaction Scheme (5), all variables are defined as in formula (I).

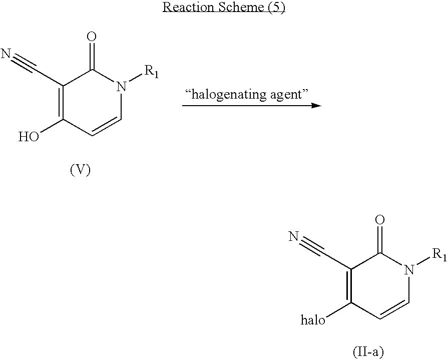

### Experimental Procedure 6

Intermediates of formula (II) wherein Y represents F₃C—S(═O)₂—O—, said intermediates being represented by formula (II-b), can be prepared by reacting an intermediate of formula (V) with triflic anhydride (also called trifloromethanesulfonic anhydride) according to Reaction Scheme (6). The reaction may be performed in a suitable reaction-inert solvent such as, for example, dichloromethane, in the presence of a suitable base such as, for example, pyridine, at a low temperature such as, for example, −78° C. In reaction scheme (6), all variables are defined as in formula (I).

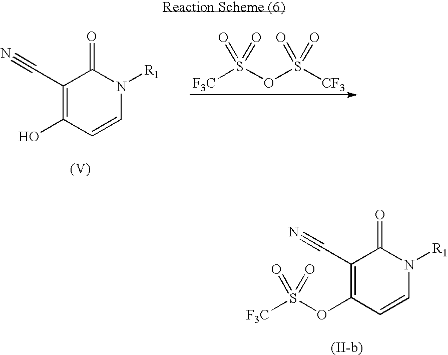

### Experimental Procedure 7

Intermediates of formula (V) can be prepared by art known procedures by reacting an intermediate of formula (VI) with a suitable reagent for methylether-cleavage, such as, for example, NaOH, according to Reaction Scheme (7). The reaction may be performed in a suitable solvent such as, for example, water at a moderately high temperature such as, for example, 100° C. In reaction scheme (6), all variables are defined as in formula (I).

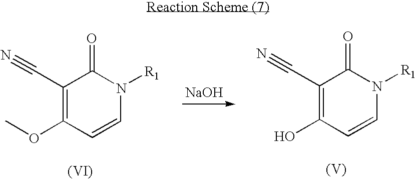

### Experimental Procedure 8

Intermediates of Formula (VI) can be prepared by art-known procedures by reacting commercially available 4-methoxy-2-oxo-1,2-dihydro-pyridine-3-carbonitrile with an alkylating agent of formula (VII), wherein Z represents a suitable leaving group, such as for example halo, e.g. bromo and the like, according to Reaction Scheme (8). An example of an alkylating agent of formula (VII) is for example cyclopropylmethylbromide. The reaction can be performed in an inert solvent such as, for example, acetonitrile, using a suitable base such as, for example, K₂CO₃, and, optionally an iodine salt such as for example KI, at a moderately high temperature such as, for example, 120° C. In reaction scheme (8), all variables are defined as in formula (I).

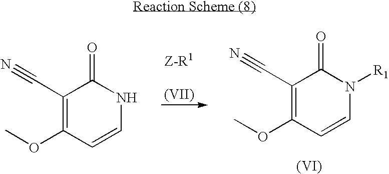

### Experimental Procedure 9

Intermediates of formula (III) can be prepared by deprotection of the piperidine nitrogen in an intermediate of formula (VIII) wherein X represents a suitable protecting group for the nitrogen atom of a piperidine derivative, such as for example tert-butoxycarbonyl, ethoxycarbonyl, benzyloxycarbonyl, benzyl and methyl, according to Reaction Scheme (9) applying art-known procedures. For example, when X represents benzyl, then the deprotection reaction may be performed in a suitable solvent, such as for example an alcohol, e.g. methanol, and 1,4 cyclohexadiene, in the presence of a suitable catalyst, such as for example palladium on charcoal, at a moderately high temperature such as, for example, 100° C. For example, when X represents an ester, the deprotection reaction can be performed by reaction with a suitable acid, such as for example hydrochloric acid, in a suitable solvent, such as for example dioxane. In reaction scheme (9), all variables are defined as in formula (I).

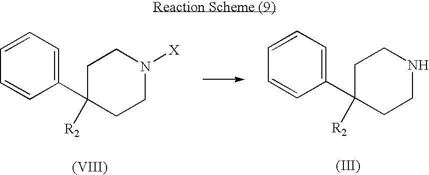

### Experimental Procedure 10

Intermediates of formula (III) wherein R₂ represents fluoro or C₁₋₄ alkyl substituted with fluoro, said R₂ being represented by -L₁-F wherein L₁ represents C₁₋₄alkyl or a covalent bond, and said intermediates being represented by formula (III-a), can be prepared by art-known procedures by reacting an intermediate of formula (IX) wherein X is a suitable protecting group for the nitrogen atom of a piperidine moiety, such as for example tert-butoxycarbonyl, ethoxycarbonyl, benzyloxycarbonyl, benzyl and methyl, with a suitable fluorinating agent such as for example (diethylamino)sulfur trifluoride [CAS: 38078-09-0], resulting in an intermediate of formula (X) according to Reaction Scheme (10) step (a). The reaction may be performed in a suitable reaction-inert solvent, such as, for example, dichloromethane, under a moderately low temperature such as, for example, a temperature ranging from −78° C. to 30° C. during for example 0.5 to 12 hours. The resulting intermediate of formula (X) can then be transformed according to Reaction Scheme (10) step (b), in an intermediate of formula (III-a) by deprotection of the piperidine nitrogen applying art-known procedures, such as for example those described in experimental procedure 9 hereinabove. In reaction scheme (10), all variables are defined as in formula (I).

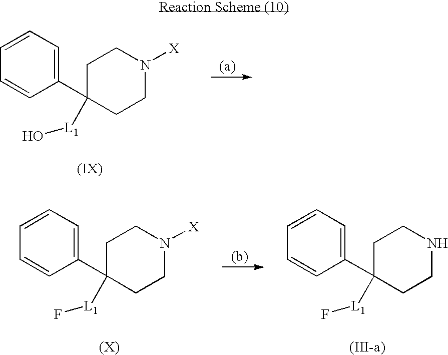

### Experimental Procedure 11

Intermediates of formula (III) wherein R₂ represents C₁₋₄alkyloxy substituted with fluoro, said C₁₋₄ alkyloxy being represented by formula Q, said R₂ being represented by -Q-F, and said intermediates being represented by formula (III-b), can be prepared by art-known procedures by reacting a hydroxyl-substituted intermediate of formula (XI) wherein X is a suitable protecting group for the nitrogen atom of a piperidine moiety, such as for example tert-butoxycarbonyl, ethoxycarbonyl, benzyloxycarbonyl, benzyl and methyl, with a suitable fluorinating agent such as (diethylamino)sulfur trifluoride [CAS: 38078-09-0], resulting in an intermediate of formula (XII) according to Reaction Scheme (11) step (a). The reaction can be performed in a suitable reaction-inert solvent, such as, for example, dichloromethane, under a moderately low temperature such as, for example, a temperature ranging from −78° C. to 30° C. during for example 0.5 to 12 hours. The intermediate of formula (XII) can then be transformed according to Reaction Scheme (11) step (b) in an intermediate of formula (III-b) by deprotection of the piperidine nitrogen applying art-known procedures, such as for example those described in experimental procedure 9 hereinabove. In reaction scheme (11), all variables are defined as in formula (I).

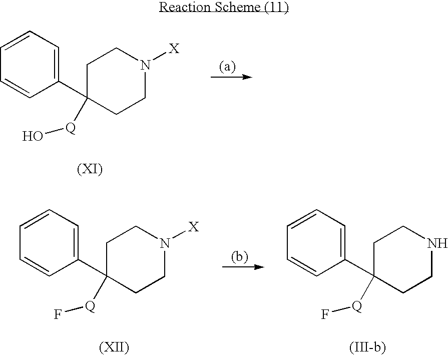

### Experimental Procedure 12

Intermediates of formula (IX) wherein L₁ represents CH₂, said intermediates being represented by formula (IX-a), can be prepared by reacting an intermediate of formula (XIII) wherein X is a suitable protecting group for the nitrogen atom of a piperidine moiety, such as for example tert-butoxycarbonyl, ethoxycarbonyl, benzyloxycarbonyl, benzyl and methyl, with a suitable reducing agent, such as for example, lithium aluminium hydride, according to Reaction Scheme (12). The reaction may be performed in a suitable solvent, such as for example tetrahydrofuran, at a moderately low temperature such as, for example, −20° C.

In reaction scheme (12), all variables are defined as in formula (I).

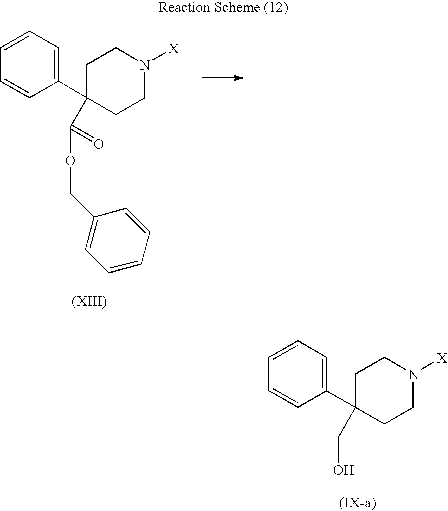

### Experimental Procedure 13

Intermediates of formula (XIV) can be prepared by reacting an intermediate of formula (XV) with fluoroC₁₋₄ alkyl-4-toluene sulfonate according to Reaction Scheme (13). The reaction can be performed in a suitable solvent, such as for example DME, and in the presence of NaH.

In reaction scheme (13), all variables are defined as in formula (I) and R'2 is defined as hereinabove.

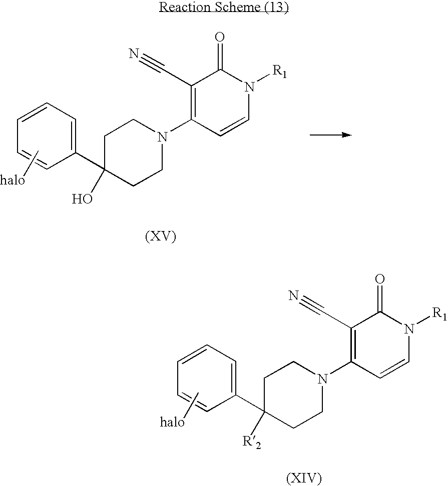

Intermediates of formula (IV) and formula (XV) also show activity as positive allosteric mGluR2 modulators. Therefore, the present invention also relates to a compound of formula (I′)

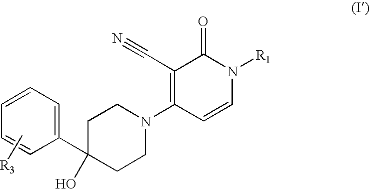

including any stereochemically isomeric form thereof, wherein
R₁ is C₄₋₆alkyl, in particular 1-butyl; or C₁₋₃ alkyl substituted with C₃₋₇ cycloalkyl, in particular cyclopropylmethyl;
R₃ is hydrogen or halo; in particular hydrogen, fluoro or chloro;
or a pharmaceutically acceptable salt thereof or a solvate thereof.

An embodiment of the present invention are those compounds of formula (I') wherein R₁ is C₄₋₆ alkyl, in particular C₄₋₅ alkyl, such as for example 1-butyl, 2-methyl-1-propyl, 3-methyl-1-butyl; in particular 1-butyl.

An embodiment of the present invention are those compounds of formula (I') wherein R₁ is C₁₋₃alkyl substituted with C₃₋₇ cycloalkyl, in particular cyclopropylmethyl or 2-(cyclopropyl)-1-ethyl.

An interesting embodiment of the compounds of formula (I') are those compounds wherein R₃ is hydrogen.

An interesting embodiment of the compounds of formula (I') are those compounds wherein R₃ is halo, in particular chloro or fluoro.

An interesting embodiment of the compounds of formula (I') are those compounds wherein R₁ is 1-butyl or cyclopropylmethyl; R₂ is hydrogen; fluoro or chloro.

Compounds of formula (I′) are for example

| | pEC₅₀ |
|---|---|
| | (see |
| | section |
| | D for |
| | pro- |
| | tocol) |
| 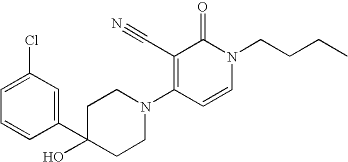 | 6.7 |
| 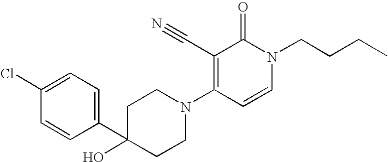 | 7.0 |
| 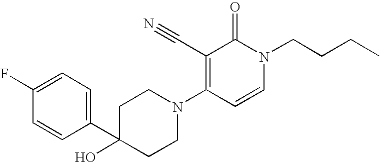 | 6.4 |
| 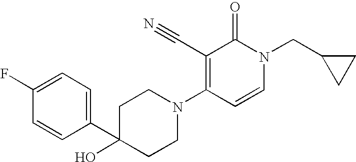 | 6.3 |
| 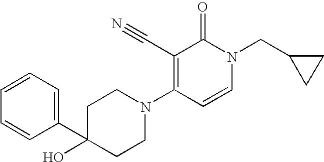 | 6.0 |

or a pharmaceutically acceptable salt thereof or a solvate thereof.

Said compounds of formula (I′) can be prepared as described hereinabove for the preparation of intermediates of formula (IV) or (XV). Reference is also made to Example A13 hereinafter.

The present invention also relates to the use of a compound of formula (I′) or any subgroup thereof for the manufacture of a medicament for treating or preventing, in particular for treating, a condition in a mammal, including a human, the treatment or prevention of which is affected or facilitated by the neuromodulatory effect of an allosteric modulator of mGluR2, in particular a positive allosteric modulator.

Since the compounds of formula (I) and formula (I′) are both positive allosteric mGluR2 modulators, the present invention also relates to a compound of formula (I″)

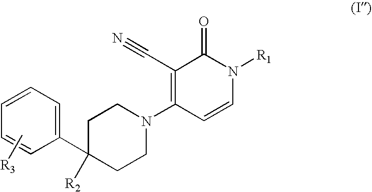

including any stereochemically isomeric form thereof, wherein
R₁ is C₄₋₆ alkyl, or C₁₋₃alkyl substituted with C₃₋₇cycloalkyl;
R₂ is hydrogen; hydroxyl; fluoro; C₁₋₄ alkyl substituted with hydroxyl; C₁₋₄ alkyl substituted with fluoro; or C₁₋₄alkyloxy substituted with fluoro;
R₃ is hydrogen or halo;
provided that if R₃ is halo, then R₂ is hydroxyl;
or a pharmaceutically acceptable salt thereof or a solvate thereof.

An embodiment of the present invention are those compounds of formula (I″) wherein R₁ is C₄₋₆ alkyl, in particular C₄₋₅ alkyl, such as for example 1-butyl, 2-methyl-1-propyl, 3-methyl-1-butyl; in particular 1-butyl.

An embodiment of the present invention are those compounds of formula (I″) wherein R₁ is C₁₋₃ alkyl substituted with C₃₋₇ cycloalkyl, in particular cyclopropylmethyl or 2-(cyclopropyl)-1-ethyl.

An embodiment of the present invention are those compounds of formula (I″) or any subgroup thereof as mentioned hereinbefore as embodiment, wherein R₂ is hydrogen.

An embodiment of the present invention are those compounds of formula (I″) or, whenever possible, any subgroup thereof as mentioned hereinbefore as embodiment, wherein R₂ is fluoro; C₁₋₄alkyl substituted with hydroxyl; C₁₋₄ alkyl substituted with fluoro; or C₁₋₄alkyloxy substituted with fluoro.

An embodiment of the present invention are those compounds of formula (I″) or, whenever possible, any subgroup thereof as mentioned hereinbefore as embodiment, wherein R₂ is hydrogen; fluoro; or C₁₋₄ alkyloxy substituted with fluoro; in particular R₂ is fluoro or C₁₋₄alkyloxy substituted with fluoro.

An embodiment of the present invention are those compounds of formula (I″) or any subgroup thereof as mentioned hereinbefore as embodiment, wherein R₂ is fluoro.

An embodiment of the present invention are those compounds of formula (I″) or, whenever possible, any subgroup thereof as mentioned hereinbefore as embodiment, wherein R₂ is C₁₋₄alkyl substituted with hydroxyl, in particular wherein R₂ is methyl substituted with hydroxyl.

An embodiment of the present invention are those compounds of formula (I″) or, whenever possible, any subgroup thereof as mentioned hereinbefore as embodiment, wherein R₂ is C₁₋₄ alkyl substituted with fluoro, in particular wherein R₂ is methyl substituted with fluoro.

An embodiment of the present invention are those compounds of formula (I″) or, whenever possible, any subgroup thereof as mentioned hereinbefore as embodiment, wherein R₂ is C₁₋₄ alkyloxy substituted with fluoro, in particular wherein R₂ is ethyloxy substituted with fluoro.

An embodiment of the present invention are those compounds of formula (I″) or, whenever possible, any subgroup thereof as mentioned hereinbefore as embodiment, wherein R₂ is hydroxyl.

The present invention also relates to the use of a compound of formula (I″) or any subgroup thereof for the manufacture of a medicament for treating or preventing, in particular for treating, a condition in a mammal, including a human, the treatment or prevention of which is affected or facilitated by the neuromodulatory effect of an allosteric modulator of mGluR2, in particular a positive allosteric modulator.

### Pharmacology

The compounds provided in this invention are positive allosteric modulators of metabotropic glutamate receptors, in particular they are positive allosteric modulators of mGluR2. The compounds of the present invention do not appear to bind to the glutamate recognition site, the orthosteric ligand site, but instead to an allosteric site within the seven transmembrane region of the receptor. In the presence of glutamate or an agonist of mGluR2, the compounds of this invention increase the mGluR2 response. The compounds provided in this invention are expected to have their effect at mGluR2 by virtue of their ability to increase the response of such receptors to glutamate or mGluR2 agonists, enhancing the response of the receptor. Hence, the present invention relates to a compound according to the present invention for use as a medicine, as well as to the use of a compound according to the invention or a pharmaceutical composition according to the invention for the manufacture of a medicament for treating or preventing, in particular treating, a condition in a mammal, including a human, the treatment or prevention of which is affected or facilitated by the neuromodulatory effect of allosteric modulators of mGluR2, in particular positive allosteric modulators thereof. The present invention also relates to a compound according to the present invention or a pharmaceutical composition according to the invention for use in the manufacture of a medicament for treating or preventing, in particular treating, a condition in a mammal, including a human, the treatment or prevention of which is affected or facilitated by the neuromodulatory effect of allosteric modulators of mGluR2, in particular positive allosteric modulators thereof. The present invention also relates to a compound according to the present invention or a pharmaceutical composition according to the invention for treating or preventing, in particular treating, a condition in a mammal, including a human, the treatment or prevention of which is affected or facilitated by the neuromodulatory effect of allosteric modulators of mGluR2, in particular positive allosteric modulators thereof.

Also, the present invention relates to the use of a compound according to the invention or a pharmaceutical composition according to the invention for the manufacture of a medicament for treating, preventing, ameliorating, controlling or reducing the risk of various neurological and psychiatric disorders associated with glutamate dysfunction in a mammal, including a human, the treatment or prevention of which is affected or facilitated by the neuromodulatory effect of positive allosteric modulators of mGluR2.

Where the invention is said to relate to the use of a compound or composition according to the invention for the manufacture of a medicament for e.g. the treatment of a mammal, it is understood that such use is to be interpreted in certain jurisdictions as a method of e.g. treatment of a mammal, comprising administering to a mammal in need of such e.g. treatment, an effective amount of a compound or composition according to the invention.

In particular, the neurological and psychiatric disorders associated with glutamate dysfunction, include one or more of the following conditions or diseases: acute neurological and psychiatric disorders such as, for example, cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including substances such as, for example, opiates, nicotine, tobacco products, alcohol, benzodiazepines, cocaine, sedatives, hypnotics, etc.), psychosis, schizophrenia, anxiety (including generalized anxiety disorder, panic disorder, and obsessive compulsive disorder), mood disorders (including depression, mania, bipolar disorders), trigeminal neuralgia, hearing loss, tinnitus, macular degeneration of the eye, emesis, brain edema, pain (including acute and chronic states, severe pain, intractable pain, neuropathic pain, and post-traumatic pain), tardive dyskinesia, sleep disorders (including narcolepsy), attention deficit/hyperactivity disorder, and conduct disorder.

In particular, the present invention relates to the use of a compound of formula (I) for the manufacture of a medicament for treating or preventing, in particular for treating, a central nervous system disorder selected from the group of anxiety disorders, psychotic disorders, personality disorders, substance-related disorders, eating disorders, mood disorders, migraine, epilepsy or convulsive disorders, childhood disorders, cognitive disorders, neurodegeneration, neurotoxicity and ischemia.

Preferably, the central nervous system disorder is an anxiety disorder, selected from the group of agoraphobia, generalized anxiety disorder (GAD), obsessive-compulsive disorder (OCD), panic disorder, posttraumatic stress disorder (PTSD), social phobia and other phobias.

Preferably, the central nervous system disorder is a psychotic disorder selected from the group of schizophrenia, delusional disorder, schizoaffective disorder, schizophreniform disorder and substance-induced psychotic disorder

Preferably, the central nervous system disorder is a personality disorder selected from the group of obsessive-compulsive personality disorder and schizoid, schizotypal disorder.

Preferably, the central nervous system disorder is a substance-related disorder selected from the group of alcohol abuse, alcohol dependence, alcohol withdrawal, alcohol withdrawal delirium, alcohol-induced psychotic disorder, amphetamine dependence, amphetamine withdrawal, cocaine dependence, cocaine withdrawal, nicotine dependence, nicotine withdrawal, opioid dependence and opioid withdrawal.

Preferably, the central nervous system disorder is an eating disorder selected from the group of anorexia nervosa and bulimia nervosa.

Preferably, the central nervous system disorder is a mood disorder selected from the group of bipolar disorders (I & II), cyclothymic disorder, depression, dysthymic disorder, major depressive disorder and substance-induced mood disorder.

Preferably, the central nervous system disorder is migraine.

Preferably, the central nervous system disorder is epilepsy or a convulsive disorder selected from the group of generalized nonconvulsive epilepsy, generalized convulsive epilepsy, petit mal status epilepticus, grand mal status epilepticus, partial epilepsy with or without impairment of consciousness, infantile spasms, epilepsy partialis continua, and other forms of epilepsy.

Preferably, the central nervous system disorder is attention-deficit/hyperactivity disorder.

Preferably, the central nervous system disorder is a cognitive disorder selected from the group of delirium, substance-induced persisting delirium, dementia, dementia due to HIV disease, dementia due to Huntington's disease, dementia due to Parkinson's disease, dementia of the Alzheimer's type, substance-induced persisting dementia and mild cognitive impairment.

Of the disorders mentioned above, the treatment of anxiety, schizophrenia, migraine, depression, and epilepsy are of particular importance.

At present, the fourth edition of the Diagnostic & Statistical Manual of Mental Disorders (DSM-IV) of the American Psychiatric Association provides a diagnostic tool for the identification of the disorders described herein. The person skilled in the art will recognize that alternative nomenclatures, nosologies, and classification systems for neurological and psychiatric disorders described herein exist, and that these evolve with medical and scientific progresses.

Because such positive allosteric modulators of mGluR2, including compounds of formula (I), enhance the response of mGluR2 to glutamate, it is an advantage that the present methods utilize endogenous glutamate.

Because positive allosteric modulators of mGluR2, including compounds of formula (I), enhance the response of mGluR2 to agonists, it is understood that the present invention extends to the treatment of neurological and psychiatric disorders associated with glutamate dysfunction by administering an effective amount of a positive allosteric modulator of mGluR2, including compounds of formula (I), in combination with an mGluR2 agonist.

The compounds of the present invention may be utilized in combination with one or more other drugs in the treatment, prevention, control, amelioration, or reduction of risk of diseases or conditions for which compounds of formula (I) or the other drugs may have utility, where the combination of the drugs together are safer or more effective than either drug alone.

### Pharmaceutical Compositions

The invention also relates to a pharmaceutical composition comprising a pharmaceutically acceptable carrier or diluent and, as active ingredient, a therapeutically effective amount of a compound according to the invention, in particular a compound according to formula (I), including a stereochemically isomeric form thereof, or a pharmaceutically acceptable salt thereof or a solvate thereof.

The compounds according to the invention, in particular the compounds according to formula (I), including a stereochemically isomeric form thereof, or a pharmaceutically acceptable salt thereof or a solvate thereof, or any subgroup or combination thereof may be formulated into various pharmaceutical forms for administration purposes. As appropriate compositions there may be cited all compositions usually employed for systemically administering drugs.

To prepare the pharmaceutical compositions of this invention, an effective amount of the particular compound, optionally in salt form, as the active ingredient is combined in intimate admixture with a pharmaceutically acceptable carrier or diluent, which carrier or diluent may take a wide variety of forms depending on the form of preparation desired for administration. These pharmaceutical compositions are desirable in unitary dosage form suitable, in particular, for administration orally, rectally, percutaneously, by parenteral injection or by inhalation. For example, in preparing the compositions in oral dosage form, any of the usual pharmaceutical media may be employed such as, for example, water, glycols, oils, alcohols and the like in the case of oral liquid preparations such as, for example, suspensions, syrups, elixirs, emulsions and solutions; or solid carriers such as, for example, starches, sugars, kaolin, diluents, lubricants, binders, disintegrating agents and the like in the case of powders, pills, capsules and tablets. Because of the ease in administration, oral administration is preferred, and tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

The exact dosage and frequency of administration depends on the particular compound of formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

As already mentioned, the invention also relates to a pharmaceutical composition comprising the compounds according to the invention and one or more other drugs in the treatment, prevention, control, amelioration, or reduction of risk of diseases or conditions for which compounds of Formula (I) or the other drugs may have utility as well as to the use of such a composition for the manufacture of a medicament. The present invention also relates to a combination of a compound according to the present invention and a mGluR2 orthosteric agonist. The present invention also relates to such a combination for use as a medicine. The present invention also relates to a product comprising (a) a compound according to the present invention, a pharmaceutically acceptable salt thereof or a solvate thereof, and (b) a mGluR2 orthosteric agonist, as a combined preparation for simultaneous, separate or sequential use in the treatment or prevention of a condition in a mammal, including a human, the treatment or prevention of which is affected or facilitated by the neuromodulatory effect of mGluR2 allosteric modulators, in particular positive mGluR2 allosteric modulators. The different drugs of such a combination or product may be combined in a single preparation together with pharmaceutically acceptable carriers or diluents, or they may each be present in a separate preparation together with pharmaceutically acceptable carriers or diluents.

The following examples are intended to illustrate but not to limit the scope of the present invention.

### Experimental Part

Several methods for preparing the compounds of this invention are illustrated in the following Examples. Unless otherwise noted, all starting materials were obtained from commercial suppliers and used without further purification. Specifically, the following abbreviations may be used in the examples and throughout the specification:

| | |
|---|---|
| EtOAc (ethyl acetate) | M (molar) |
| LCMS (Liquid Chromatography Mass | MeOH (methanol) |
| Spectrum) | g (grams) |
| DCM (dichloromethane) | min (minutes) |
| ml (millilitres) | DMF (dimethylformamide) |
| mmol (millimol) | THF (tetrahydrofuran) |
| P(═O)Br₃ (phosphoric tribromide) | HPLC (High Pressure Liquid |
| Et₂O (diethyl ether) | Chromatography) |
| DME (dimethoxyethane) | |

All references to brine refer to a saturated aqueous solution of NaCl. Unless otherwise indicated, all temperatures are expressed in ° C. (degrees Celsius). All reactions are conducted not under an inert atmosphere at room temperature, unless otherwise noted.

Microwave assisted reactions were performed in a single-mode reactor: Emrys™ Optimizer microwave reactor (Personal Chemistry A.B., currently Biotage) or in a multimode reactor: MicroSYNTH Labstation (Milestone, Inc.).

### A. Preparation of the Intermediates

### Example A.1

### 1-Cyclopropylmethyl-4-methoxy-2-oxo-1,2-dihydro-pyridine-3-carbonitrile (Intermediate 1)

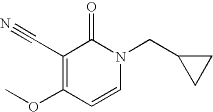

To a solution of 4-methoxy-2-oxo-1,2-dihydro-pyridine-3-carbonitrile (12.2 g, 81.48 mmol) in acetonitrile (250 ml) were added bromomethyl-cyclopropane (11 g, 81.48 mmol) and potassium carbonate (22.48 g, 162.9 mmol) and the mixture was heated at 110° C. for 24 hours. The mixture was cooled to room temperature and the solid was filtered off. The filtrate was evaporated till dryness and the resulting crude residue was then triturated with diethylether to yield pure intermediate 1 (15.72 g, 94%) as a white solid.

### Example A.2

### 1-Butyl-4-methoxy-2-oxo-1,2-dihydro-pyridine-3-carbonitrile (Intermediate 2)

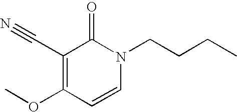

To a solution of 4-methoxy-2-oxo-1,2-dihydro-pyridine-3-carbonitrile (20 g, 133 mmol) in acetonitrile (800 ml) were added 1-bromobutane (15.8 ml, 146 mmol) and potassium carbonate (36.7 g, 266 mmol) and the mixture was heated at 110° C. for 24 hours. The mixture was cooled to room temperature and the solid was filtered off. The filtrate was evaporated till dryness and the resulting crude residue was then triturated with diethylether to yield pure intermediate 2 (27.39 g, >99%) as a white solid.

### Example A.3

### 1-Cyclopropylmethyl-4-hydroxy-2-oxo-1,2-dihydro-pyridine-3-carbonitrile (Intermediate 3)

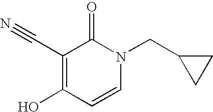

Intermediate 1 (15.7 g, 76.8 mmol) was added at room temperature to a 1N aqueous solution of sodium hydroxide (300 ml) and THF (50 ml). The reaction mixture was heated at 140° C. (oil bath temperature) for 16 hours. The mixture was cooled to room temperature and the THF was mostly evaporated in vacuo. The aqueous layer was cooled to 0° C. and acidified by the addition of aqueous 2N HCl, adjusting the pH to about 3, at which point a white solid precipitated. The solid was filtered off, washed with Et₂O and dried in vacuo to yield intermediate 3 as a white solid (10.44 g, 71%) that was used without further purification.

### Example A.4

### 1-Butyl-4-hydroxy-2-oxo-1,2-dihydro-pyridine-3-carbonitrile (Intermediate 4)

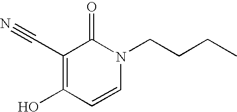

Intermediate 2 (27.39 g, 133 mmol) was added at room temperature to a 1N aqueous solution of sodium hydroxide (500 ml) and THF (100 ml). The reaction mixture was heated at 110° C. (oil bath temperature) for 24 hours. The mixture was cooled to room temperature and the solvent was evaporated in vacuo until the volume was reduced to approximately 250 ml. The aqueous layer was then cooled to 0° C. and acidified by the addition of aqueous 2N HCl, adjusting the pH to about 3, at which point a white solid precipitated. The solid was filtered off, washed with Et₂O and dried in vacuo to yield intermediate 4 as a white solid (25 g, 98%) that was used without further purification.

### Example A.5

### 4-Bromo-1-cyclopropylmethyl-2-oxo-1,2-dihydro-pyridine-3-carbonitrile (Intermediate 5)

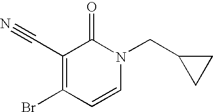

To a solution of intermediate 3 (10.4 g, 54.67 mmol) in DMF (250 ml) was added P(═O)Br₃ (31.3 g, 109.3 mmol) and the mixture was heated at 110° C. for 1.5 hours. After cooling in an ice bath the solution was partitioned between water and EtOAc. After three extractions with EtOAc the combined organic fractions were washed with brine, dried over MgSO₄ and the solvent evaporated in vacuo. The crude product was purified by column chromatography (silica gel; DCM as eluent). The desired fractions were collected and evaporated in vacuo to yield intermediate 5 (8.83 g, 64%).

### Example A.6

### 4-Bromo-1-butyl-2-oxo-1,2-dihydro-pyridine-3-carbonitrile (Intermediate 6)

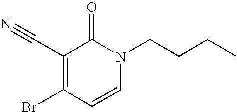

To a solution of intermediate 4 (39 g, 203 mmol) in DMF (600 ml) was added P(═O)Br₃ (116 g, 406 mmol) and the mixture was heated at 110° C. for 1.5 hours. After cooling in an ice bath the solution was partitioned between water and EtOAc. After three extractions with EtOAc the combined organic fractions were washed with brine, dried over Na₂SO₄ and the solvent evaporated in vacuo. The crude product was purified by column chromatography (silica gel; DCM as eluent). The desired fractions were collected and evaporated in vacuo to yield intermediate 6 (36.7 g, 72%).

### Example A.7

### 4-Hydroxy-4-phenyl-piperidine-1-carboxylic acid tert-butyl ester (Intermediate 7)

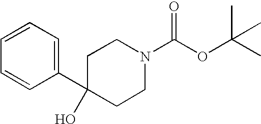

To a solution of 4-hydroxy-4-phenylpiperidine (2 g, 11.28 mmol) in dichloromethane (50 ml), at room temperature, di-tent-butyl dicarbonate (2.95 g, 13.53 mmol) was added. The resulting mixture was stirred at room temperature for 5 hours. The volatiles were evaporated in vacuo to yield crude intermediate 7, used without further purification (3.12 g, 100%).

### Example A.8

### 4-Fluoro-4-phenyl-piperidine-1-carboxylic acid tert-butyl ester (Intermediate 8)

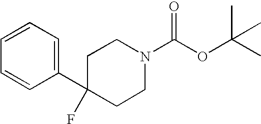

Reaction carried out under nitrogen atmosphere. To a solution of (diethylamino)sulfur trifluoride (0.74 ml, 5.67 mmol) in dichloromethane (30 ml) previously cooled to −78° C., was added a solution of intermediate 7 (1.5 g, 5.4 mmol) in dichloromethane (30 ml) dropwise. The resulting mixture was then stirred at −78° C. for 1 hour and then the reaction mixture was allowed to warm to room temperature and further stirred for 30 minutes. NaHCO₃ (aqueous saturated solution, 90 ml) was added and the mixture was stirred for 15 minutes. The organic layer was separated and treated with 3-chloroperoxybenzoic acid (0.2 g, 1.18 mmol) and stirred for 30 minutes. The reaction mixture was washed successively with NaHCO₃ (aqueous saturated solution), water and brine. The organic layer was dried over Na₂SO₄ and evaporated in vacuo to yield crude intermediate 8 used without further purification (1.5 g, 100%).

### Example A.9

### 4-Fluoro-4-phenyl-piperidine hydrochloride (Intermediate 9)

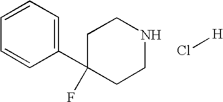

Intermediate 8 (1.5 g, 5.37 mmol) was dissolved in hydrochloric acid (4N in 1,4-dioxane, 20 ml) and the resulting solution was stirred at room temperature for 2 hours.

The volatiles were evaporated in vacuo and the residue thus obtained was treated with diethylether to yield intermediate 9 as a solid (1.15 g, 100%; .HCl).

### Example A. 10

### 1-Benzyl-4-phenyl-piperidine-4-carboxylic acid benzyl ester (Intermediate 10)

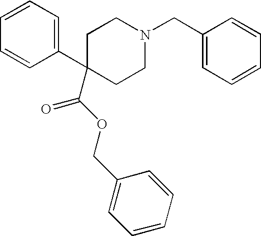

4-Phenyl-4-piperidinecarboxylic acid 4-methylbenzenesulfonate (CAS83949-32-0) (2 g, 5.3 mmol), benzylbromide (0.76 ml, 6.36 mmol) and potassium carbonate (2.92 g, 21.2 mmol) were suspended in acetonitrile (6 ml) and heated at 130° C. for 15 minutes under microwave irradiation. The cooled reaction was filtered through a pad of diatomaceous earth. The pad of diatomaceous earth was washed with acetonitrile, EtOAc and dichloromethane. The combined organic filtrates were evaporated in vacuo to yield crude intermediate 10 (2.04 g, 100%) used without further purification.

### Example A.11

### (1-Benzyl-4-phenyl-piperidin-4-yl)-methanol (Intermediate 11)

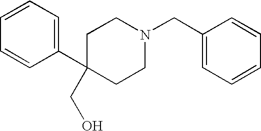

Reaction carried out under nitrogen atmosphere. Intermediate 10 (2.04 g, 5.3 mmol) was suspended in dry tetrahydrofuran (25 ml) and the mixture was cooled to −78° C. Lithium aluminum hydride (1M in tetrahydrofuran, 7.95 ml, 7.95 mmol) was dropwise added. The resulting reaction mixture was gradually warmed to room temperature and further stirred for 2 hours. NH₄Cl (aqueous saturated solution) was added and the reaction mixture was extracted first with EtOAc and then with 1-hydroxybutane. The combined organic extracts were dried over Na₂SO₄ and evaporated in vacuo to yield crude intermediate 11 (0.65 g, 43%) used without further purification.

### Example A.12

### (4-Phenyl-piperidin-4-yl)-methanol (Intermediate 12)

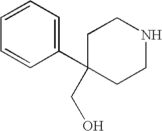

Reaction carried out under nitrogen atmosphere. Intermediate 11 (0.65 g, 2.31 mmol) was suspended in methanol (10 ml) and then 1,4-cyclohexadiene (2.17 ml, 23.1 mmol) and palladium 10% activated on carbon (0.65 g) were added. The resulting mixture was heated at 100° C. for 24 hours into a sealed tube. The cooled reaction mixture was filtered through a pad of diatomaceous earth. The pad of diatomaceous earth was washed successively with methanol and a 7N solution of methanol saturated with ammonia. The combined organic filtrates were evaporated in vacuo to yield crude intermediate 12 (0.47 g, 100%) used without further purification.

### Example A.13

### 1′-Butyl-4-hydroxy-2′-oxo-4-(4-chlorophenyl)-3,4,5,6,1′,2′-hexahydro-2H-[1,4′]bipyridinyl-3′-carbonitrile (Intermediate 13)

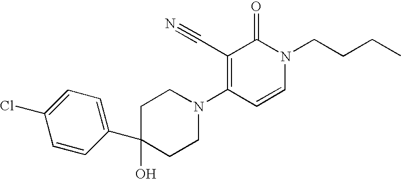

A mixture of intermediate 6 (0.255 g, 1.39 mmol), 4-(4-chlorophenyl)-4-hydroxypiperidine (C.A.S. 39512-49-7) (0.265 g, 1.25 mmol) and N,N-diisopropylethylamine (0.348 ml, 2 mmol) in acetonitrile (2.5 ml) was microwaved at 150° C. for 10 minutes. The reaction mixture was diluted with NaHCO₃ (aqueous saturated solution) and extracted with DCM. The organic layer was dried over Na₂SO₄ and evaporated in vacuo. The crude product was purified by column chromatography (silica gel; DCM to DCM/EtOAc 9:1 to 1:9). The desired fractions were collected and evaporated in vacuo to yield intermediate 13 (0.357 g, 93%). 1′-Butyl-4-hydroxy-2′-oxo-4-(3-chlorophenyl)-3,4,5,6,1′,2′-hexahydro-2H-[1,4′]bipyridinyl-3′-carbonitrile (Intermediate 13a)

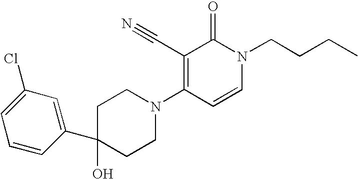

Intermediate 13a was prepared according to the protocol for intermediate 13 but starting from 4-(3-chlorophenyl)-4-hydroxypiperidine.

### Example A.14

### 1′-Butyl-4-fluoroethyloxy-2′-oxo-4-(4-chlorophenyl)-3,4,5,6,1′,2′-hexahydro-2H-[1,4′]bipyridinyl-3′-carbonitrile (Intermediate 14)

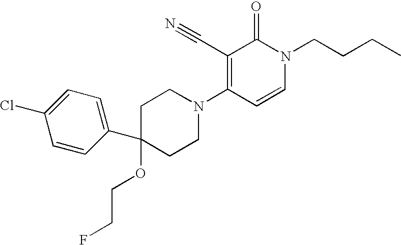

To NaH (0.020 g, 0.5 mmol, 60% in mineral oil) at 0° C., fluoroethyl 4-toluenesulfonate (C.A.S. 383-50-3) (0.063 g, 0.029 mmol) in DME (2 ml) was dropwise added. The mixture was stirred at room temperature for 15 minutes. Then, intermediate 13 (0.1 g, 0.26 mmol) in DME (1 ml) was added and the reaction was microwaved at 150° C. for 15 minutes. Then NH₄Cl (aqueous saturated solution) was added. The resulting mixture was extracted with AcOEt. The organic layer was dried over Na₂SO₄ and evaporated in vacuo. The crude product was purified by column chromatography (silica gel; DCM to DCM/EtOAc 9:1). The desired fractions were collected and evaporated in vacuo to yield intermediate 14 (0.038 g, 34%).

To prepare 1′-butyl-4-fluoroethyloxy-2′-oxo-4-(3-chlorophenyl)-3,4,5,6,1′,2′-hexahydro-2H-[1,4]bipyridinyl-3′-carbonitrile (intermediate 14a), intermediate 13a is reacted according to the protocol for the preparation of intermediate 14.

### B. Preparation of the Final Compounds

### Example B.1

### 1′-Cyclopropylmethyl-4-fluoro-2′-oxo-4-phenyl-3,4,5,6,1′,2′-hexahydro-2H-[1,4′]bipyridinyl-3′-carbonitrile (Compound 1)

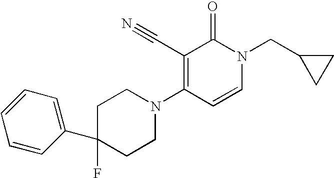

A mixture of intermediate 5 (1.0 g, 4.84 mmol), intermediate 9 (1.15 g, 5.33 mmol) and N,N-diisopropylethylamine (3.3 ml, 19.36 mmol) in acetonitrile (3 ml) was microwaved at 150° C. for 15 minutes. The mixture was cooled to room temperature and the solvents were evaporated in vacuo. The crude product was purified by column chromatography (silica gel; DCM to DCM/MeOH(NH₃) up to 5% as eluent). The desired fractions were collected and evaporated in vacuo to yield compound 1 as a pale yellow solid (0.874 g, 51%)

### Example B.2

### 1′-Butyl-4-fluoro-2′-oxo-4-phenyl-3,4,5,6,1′,2′-hexahydro-2H-[1,4′]bipyridinyl-3′-carbonitrile (Compound 2)

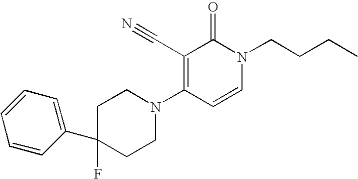

A mixture of intermediate 6 (0.354 g, 1.39 mmol), intermediate 9 (0.3 g, 1.39 mmol) and N,N-diisopropylethylamine (0.72 ml, 4.17 mmol) in acetonitrile (3 ml) was microwaved at 150° C. for 15 minutes. The mixture was cooled to room temperature and the solvents were evaporated in vacuo. The crude product was purified by column chromatography (silica gel; DCM to DCM/MeOH(NH₃) up to 10% as eluent). The desired fractions were collected and evaporated in vacuo to yield compound 2 as a pale pink solid (0.104 g, 21%).

### Example B.3

### 1′-Cyclopropylmethyl-4-hydroxymethyl-2′-oxo-4-phenyl-3,4,5,6,1′,2′-hexahydro-2H-[1,4′]bipyridinyl-3′-carbonitrile (Compound 3)

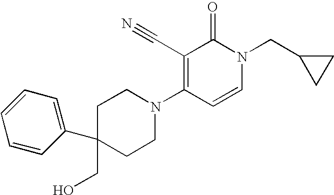

A mixture of intermediate 5 (0.32 g, 1.3 mmol), intermediate 12 (0.25 g, 1.3 mmol) and N,N-diisopropylethylamine (0.45 ml, 2.6 mmol) in acetonitrile (3 ml) was microwaved at 150° C. for 15 minutes. The mixture was cooled to room temperature and the solvents were evaporated in vacuo. The crude product was purified by column chromatography (silica gel; DCM to DCM/MeOH(NH₃) up to 10% as eluent). The desired fractions were collected and evaporated in vacuo to yield compound 3 as an oil (0.385 g, 81%).

### Example B.4

### 1′-Butyl-4-hydroxymethyl-2′-oxo-4-phenyl-3,4,5,6,1′,2′-hexahydro-2H-[1,4′]bipyridinyl-3′-carbonitrile (Compound 4)

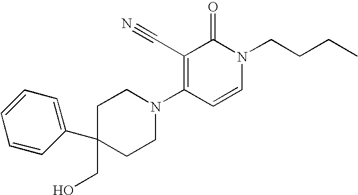

A mixture of intermediate 6 (0.267 g, 1.04 mmol), intermediate 12 (0.2 g, 1.04 mmol) and N,N-diisopropylethylamine (0.54 ml, 3.12 mmol) in acetonitrile (2 ml) was microwaved at 150° C. for 15 minutes. The mixture was cooled to room temperature and the solvents were evaporated in vacuo. The crude product was purified by column chromatography (silica gel; DCM to DCM/MeOH(NH₃) up to 10% as eluent). The desired fractions were collected and evaporated in vacuo to yield compound 4 as a pale brown solid (0.100 g, 26%).

### Example B.5

### 1′-Cyclopropylmethyl-4-fluoromethyl-2′-oxo-4-phenyl-3,4,5,6,1′,2′-hexahydro-2H-[1,4′]bipyridinyl-3′-carbonitrile (Compound 5)

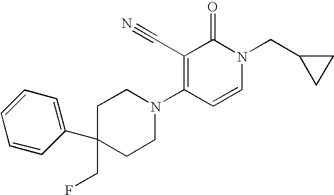

Reaction carried out under nitrogen atmosphere. To a solution of (diethylamino)sulfur trifluoride (0.145 ml, 1.11 mmol) in dichloromethane (30 ml) previously cooled to −78° C., was added a solution of compound 3 (0.385 g, 1.06 mmol) in dichloromethane (30 ml) dropwise. The resulting mixture was then stirred at −78° C. for 1 hour and then the reaction mixture was allowed to warm to room temperature and further stirred for 30 minutes. NaHCO₃ (aqueous saturated solution, 90 ml) was added and the mixture was stirred for 15 minutes. The organic layer was separated and treated with 3-chloroperoxybenzoic acid (0.047 g, 0.27 mmol) and stirred for 30 minutes. The reaction mixture was washed successively with NaHCO₃ (aqueous saturated solution), water and brine. The organic layer was dried over Na₂SO₄ and evaporated in vacuo.

The crude product was purified by reversed phase preparative HPLC to yield compound 5 (0.112 g, 29%).

### Example B.6

### 1′-Butyl-4-fluoromethyl-2′-oxo-4-phenyl-3,4,5,6,1′,2′-hexahydro-2H-[1,4′]bipyridinyl-3′-carbonitrile (Compound 6)

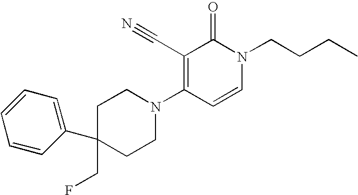

Reaction carried out under nitrogen atmosphere. To a solution of (diethylamino)sulfur trifluoride (0.075 ml, 0.57 mmol) in dichloromethane (15 ml) previously cooled to −78° C., was added a solution of compound 4 (0.20 g, 0.54 mmol) in dichloromethane (15 ml) dropwise. The resulting mixture was then stirred at −78° C. for 1 hour and then the reaction mixture was allowed to warm to room temperature and further stirred for 30 minutes. NaHCO₃ (aqueous saturated solution, 90 ml) was added and the mixture was stirred for 15 minutes. The organic layer was separated and treated with 3-chloroperoxybenzoic acid (0.024 g, 0.14 mmol) and stirred for 30 minutes. The reaction mixture was washed successively with NaHCO₃ (aqueous saturated solution), water and brine. The organic layer was dried over Na₂SO₄ and evaporated in vacuo. The crude product was purified by column chromatography (silica gel; DCM to DCM/EtOAc up to 20% as eluent). The desired fractions were collected and evaporated in vacuo to yield compound 6 as a white solid (0.035 g, 17%).

### Example B.7

### 1′-Butyl-4-fluoroethyloxy-2′-oxo-4-phenyl-3,4,5,6,1′,2′-hexahydro-2H-[1,4′]bipyridinyl-3′-carbonitrile (Compound 7)

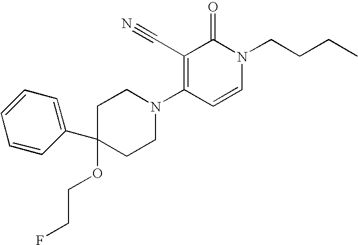

A solution of intermediate 14 (0.038 g, 0.09 mmol) in methanol (2 ml) and triethylamine (0.025 ml, 0.18 mmol) was hydrogenated at room temperature in the presence of palladium 10% on activated carbon (0.005 g) for 2 hours. The solids were filtered off and the filtrate was evaporated till dryness. The crude reaction mixture was then purified by column chromatography (silica gel; DCM to DCM/MeOH up to 1% as eluent). The desired fractions were collected and evaporated in vacuo yielding compound 7 (0.019 g, 53%).

An alternative way to prepare compound 7 is to react intermediate 14a according to the above protocol.

### Example B.8

### 3-cyano-1-cyclopropylmethyl-4-(4-phenyl-piperidin-1-yl)-pyridine-2(1H)-one (Compound 8)

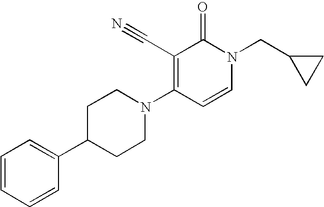

A mixture of intermediate 5 (0.3 g, 1.18 mmol), 4-phenylpiperidine (0.286 g, 1.77 mmol) and diisopropylethylamine (0.615 ml, 3.54 mmol) in acetonitrile (5 ml) was microwaved at 150° C. for 20 minutes. The mixture was cooled to room temperature and the solvents were evaporated in vacuum. The residue thus obtained was purified by flash chromatography (SiO₂, DCM/MeOH(NH₃) mixtures) to yield the desired compound. The compound was then recrystallised from ethylether to yield compound 8 (0.29 g, 73%)

Compound 9 was prepared according to the protocol described for compound 8.

Table 1 lists compounds of formula (I) that were prepared according to one of the above Examples (Ex. no.).

TABLE 1

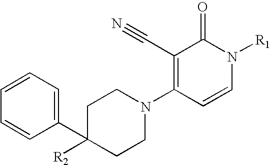

| Comp. no. | Ex. no. | 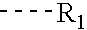 | 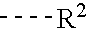 |
|---|---|---|---|
| 1 | B1 | 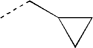 | 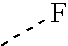 |
| 2 | B2 | 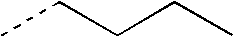 | 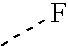 |
| 3 | B3 | 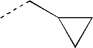 | 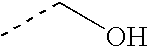 |
| 4 | B4 | 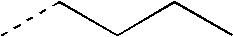 | 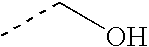 |
| 5 | B5 | 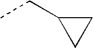 | 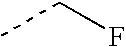 |
| 6 | B6 | 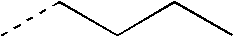 | 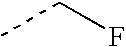 |
| 7 | B7 | 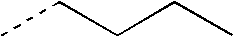 | 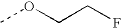 |
| 8 | B8 | 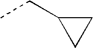 | H |
| 9 | B8 | 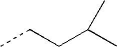 | H |

### C. Analytical Part

For LCMS-characterization of the compounds of the present invention, the following method was used.

### LCMS—General Procedure

The HPLC measurement was performed using a HP 1100 from Agilent Technologies comprising a pump (quaternary or binary) with degasser, an autosampler, a column oven, a diode-array detector (DAD) and a column as specified in the respective methods below. Flow from the column was split to a MS detector. The MS detector was configured with an electrospray ionization source. Nitrogen was used as the nebulizer gas. The source temperature was maintained at 140° C. Data acquisition was performed with MassLynx-Openlynx software.

### Method 1

In addition to the general procedure: Reversed phase HPLC was carried out on an XDB-C18 cartridge (1.8 μm, 2.1×30 mm) from Agilent, with a flow rate of 1 ml/min, at 60° C. The gradient conditions used are: 90% A (0.5 g/l ammonium acetate solution), 5% B (acetonitrile), 5% C (methanol) to 50% B and 50% C in 6.5 minutes, to 100% B at 7 minutes and equilibrated to initial conditions at 7.5 minutes until 9.0 minutes. Injection volume 2 μl. High-resolution mass spectra (Time of Flight, TOF) were acquired only in positive ionization mode by scanning from 100 to 750 in 0.5 seconds using a dwell time of 0.1 seconds. The capillary needle voltage was 2.5 kV and the cone voltage was 20 V. Leucine-Enkephaline was the standard substance used for the lock mass calibration.

### Method 2:

In addition to the general procedure: Reversed phase HPLC was carried out on a LUNA-C18 column (2.5 μm, 2.1×30 mm) from Phenomenex, with a flow rate of 1.0 ml/min, at 60° C. The gradient conditions used are: 90% A (0.5 g/l ammonium acetate solution), 5% B (acetonitrile), 5% C (methanol) to 50% B and 50% C in 6.5 minutes, to 100% B at 7 minutes and equilibrated to initial conditions at 7.5 minutes until 9.0 minutes. Injection volume 5 μl. High-resolution mass spectra (Time of Flight, TOF) were acquired only in positive ionization mode by scanning from 100 to 750 in 0.5 seconds using a dwell time of 0.1 seconds. The capillary needle voltage was 2.5 kV for positive ionization mode and the cone voltage was 20 V. Leucine-Enkephaline was the standard substance used for the lock mass calibration.

### Method 3:

In addition to the general procedure: Reversed phase HPLC was carried out on an ACE-C18 column (3.0 μm, 4.6×30 mm) from Advanced Chromatography Technologies, with a flow rate of 1.5 ml/min, at 40° C. The gradient conditions used are: 80% A (0.5 g/l ammonium acetate solution), 10% B (acetonitrile), 10% C (methanol) to 50% B and 50% C in 6.5 minutes, to 100% B at 7 minutes and equilibrated to initial conditions at 7.5 minutes until 9.0 minutes. Injection volume 5 μl. High-resolution mass spectra (Time of Flight, TOF) were acquired only in positive ionization mode by scanning from 100 to 750 in 0.5 seconds using a dwell time of 0.1 seconds. The capillary needle voltage was 2.5 kV for positive ionization mode and the cone voltage was 20 V. Leucine-Enkephaline was the standard substance used for the lock mass calibration.

### Melting Point Determination

Melting point determinations were performed on a Mettler FP62 apparatus.

TABLE 2
Analytical data (Rₜ means retention time in minutes; (MH)⁺
means the protonated mass of the compound (free base).

| | Melting | | | |
|---|---|---|---|---|
| Comp. No. | Point (° C.) | MH+ | Rₜ (min) | Method |
| Compound 1 | 189.2 | 352 | 3.98 | 1 |
| Compound 2 | n.d. | 354 | 4.27 | 1 |
| Compound 3 | n.d. | 364 | 3.29 | 1 |
| Compound 4 | 103.2 | 366 | 3.58 | 1 |
| Compound 5 | decomposes | 366 | 4.19 | 1 |
| Compound 6 | n.d. | 368 | 4.45 | 1 |
| Compound 7 | n.d. | 398 | 4.45 | 2 |
| Compound 8 | 128 | 334 | 4.05 | 1 |
| Compound 9 | decomposes | 350 | 4.76 | 3 |

n.d.: not determined

### D. Pharmacological Examples

The compounds provided in the present invention are positive allosteric modulators of mGluR2. These compounds appear to potentiate glutamate responses by binding to an allosteric site other than the glutamate binding site. The response of mGluR2 to a concentration of glutamate is increased when compounds of formula (I) are present. Compounds of formula (I) are expected to have their effect substantially at mGluR2 by virtue of their ability to enhance the function of the receptor. The behaviour of positive allosteric modulators tested at mGluR2 using the [³⁵S]GTPγS binding assay method described below and which is suitable for the identification of such compounds, and more particularly the compounds according to formula (I), are shown in Table 3.

### [³⁵S]GTPγS Binding Assay

The [³⁵S]GTPγS binding assay is a functional membrane-based assay used to study G-protein coupled receptor (GPCR) function whereby incorporation of a non-hydrolysable form of GTP, [³⁵S]GTPγS (guanosine 5′-triphosphate, labelled with gamma-emitting ³⁵S), is measured. The G-protein γ subunit catalyzes the exchange of guanosine 5′-diphosphate (GDP) by guanosine triphosphate (GTP) and on activation of the GPCR by an agonist, [³⁵S]GTPγS, becomes incorporated and cannot be cleaved to continue the exchange cycle (Harper (1998) Current Protocols in Pharmacology 2.6.1-10, John Wiley & Sons, Inc.). The amount of radioactive [³⁵S]GTPγS incorporation is a direct measure of the activity of the G-protein and hence the activity of the agonist can be determined. mGluR2 receptors are shown to be preferentially coupled to Gγi-protein, a preferential coupling for this method, and hence it is widely used to study receptor activation of mGluR2 receptors both in recombinant cell lines and in tissues (Schaffhauser et al 2003, Pinkerton et al, 2004, Mutel et al (1998) Journal of Neurochemistry. 71:2558-64; Schaffhauser et al (1998) Molecular Pharmacology 53:228-33). Here we describe the use of the [³⁵S]GTPγS binding assay using membranes from cells transfected with the human mGluR2 receptor and adapted from Schaffhauser et al ((2003) Molecular Pharmacology 4:798-810) for the detection of the positive allosteric modulation (PAM) properties of the compounds of this invention.

### Membrane Preparation

CHO-cells were cultured to pre-confluence and stimulated with 5 mM butyrate for 24 hours, prior to washing in PBS, and then collection by scraping in homogenisation buffer (50 mM Tris-HCl buffer, pH 7.4, 4° C.). Cell lysates were homogenized briefly (15s) using an ultra-turrax homogenizer. The homogenate was centrifuged at 23 500×g for 10 minutes and the supernatant discarded. The pellet was resuspended in 5 mM Tris-HCl, pH 7.4 and centrifuged again (30 000×g, 20 min, 4° C.). The final pellet was resuspended in 50 mM HEPES, pH 7.4 and stored at −80° C. in appropriate aliquots before use. Protein concentration was determined by the Bradford method (Bio-Rad, USA) with bovine serum albumin as standard.

### [³⁵S]GTPγS Binding Assay

Measurement of mGluR2 positive allosteric modulatory activity of test compounds in membranes containing human mGluR2 was performed using frozen membranes that were thawed and briefly homogenised prior to pre-incubation in 96-well microplates (15 μg/assay well, 30 minutes, 30° C.) in assay buffer (50 mM HEPES pH 7.4, 100 mM NaCl, 3 mM MgCl₂, 50 μM GDP, 10 μg/ml saponin,) with increasing concentrations of positive allosteric modulator (from 0.3 nM to 50 μM) and either a minimal pre-determined concentration of glutamate (PAM assay), or no added glutamate. For the PAM assay, membranes were pre-incubated with glutamate at EC₂₅ concentration, i.e. a concentration that gives 25% of the maximal response glutamate, and is in accordance to published data (Pin et al. (1999) Eur. J. Pharmacol. 375:277-294). After addition of [³⁵S]GTPγS (0.1 nM, f.c.) to achieve a total reaction volume of 200 μl, microplates were shaken briefly and further incubated to allow [³⁵S]GTPγS incorporation on activation (30 minutes, 30° C.). The reaction was stopped by rapid vacuum filtration over glass-fibre filter plates (Unifilter 96-well GF/B filter plates, Perkin-Elmer, Downers Grove, USA) microplate using a 96-well plate cell harvester (Filtermate, Perkin-Elmer, USA), and then by washing three times with 300 μl of ice-cold wash buffer (Na₂PO₄.2H₂0 10 mM, NaH₂PO₄.H₂0 10 mM, pH=7.4). Filters were then air-dried, and 40 μl of liquid scintillation cocktail (Microscint-O) was added to each well, and membrane-bound [³⁵S]GTPγS was measured in a 96-well scintillation plate reader (Top-Count, Perkin-Elmer, USA). Non-specific [³⁵S]GTPγS binding is determined in the presence of cold 10 μM GTP. Each curve was performed at least once using duplicate sample per data point and at 11 concentrations.

### Data Analysis

The concentration-response curves of representative compounds of the present invention in the presence of added EC₂₅ of mGluR2 agonist glutamate to determine positive allosteric modulation (PAM), were generated using the Prism GraphPad software (Graph Pad Inc, San Diego, USA). The curves were fitted to a four-parameter logistic equation (Y=Bottom+(Top-Bottom)/(1+10̂((LogEC₅₀-X)*Hill Slope) allowing determination of EC₅₀ values. The EC₅₀ is the concentration of a compound that causes a half-maximal potentiation of the glutamate response. This is calculated by subtracting the maximal responses of glutamate in presence of a fully saturating concentration of a positive allosteric modulator from the response of glutamate in absence of a positive allosteric modulator. The concentration producing the half-maximal effect is then calculated as EC₅₀.

TABLE 3
Pharmacological data for compounds according to the invention.

| | GTPgS - hR2 |
|---|---|
| Comp. No. | PAM pEC₅₀ |
| 1 | 6.58 |
| 2 | 6.76 |
| 3 | nm |
| 4 | 5.69 |
| 5 | 5.92 |
| 6 | 6.35 |
| 7 | 6.73 |
| 8 | 6.5 |
| 9 | 7.2 |

nm: not measured
All compounds were tested in presence of mGluR2 agonist, glutamate at a predetermined EC₂₅ concentration, to determine positive allosteric modulation (GTPγS-PAM). Values shown are averages of duplicate values of 11-concentration response curves, from at least one experiment. All tested compounds showed a pEC₅₀ (-logEC₅₀) value of more than 5.0. The error of determination of a pEC₅₀ value for a single experiment is estimated to be about 0.3 log-units.

### E. Composition Examples

“Active ingredient” as used throughout these examples relates to a final compound of formula (I), the pharmaceutically acceptable salts thereof, the solvates and the stereochemically isomeric forms thereof.

Typical examples of recipes for the formulation of the invention are as follows:

### 1. Tablets

| | |
|---|---|
| Active ingredient | 5 to 50 mg |
| Di-calcium phosphate | 20 mg |
| Lactose | 30 mg |
| Talcum | 10 mg |
| Magnesium stearate | 5 mg |
| Potato starch | ad 200 mg |

In this Example, active ingredient can be replaced with the same amount of any of the compounds according to the present invention, in particular by the same amount of any of the exemplified compounds.

### 2. Suspension

An aqueous suspension is prepared for oral administration so that each 1 milliliter contains 1 to 5 mg of one of the active compounds, 50 mg of sodium carboxymethyl cellulose, 1 mg of sodium benzoate, 500 mg of sorbitol and water ad 1 ml.

### 3. Injectable

A parenteral composition is prepared by stirring 1.5% by weight of active ingredient of the invention in 10% by volume propylene glycol in water.

### 4. Ointment

| | |
|---|---|
| Active ingredient | 5 to 1000 mg |
| Stearyl alcohol | 3 g |
| Lanoline | 5 g |
| White petroleum | 15 g |
| Water | ad 100 g |

In this Example, active ingredient can be replaced with the same amount of any of the compounds according to the present invention, in particular by the same amount of any of the exemplified compounds.

Reasonable variations are not to be regarded as a departure from the scope of the invention. It will be obvious that the thus described invention may be varied in many ways by those skilled in the art.

1. A compound of formula (I″) 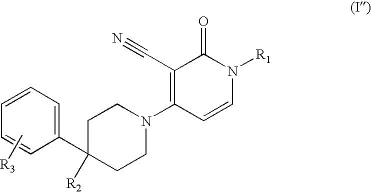
including any stereochemically isomeric form thereof, wherein
R₁ is C₄₋₆ alkyl, or C₁₋₃ alkyl substituted with C₃₋₇ cycloalkyl;
R₂ is hydrogen; hydroxyl; fluoro; C₁₋₄ alkyl substituted with hydroxyl; C₁₋₄ alkyl substituted with fluoro; or C₁₋₄alkyloxy substituted with fluoro;
R₃ is hydrogen or halo;
provided that if R₃ is halo, then R₂ is hydroxyl;
or a pharmaceutically acceptable salt thereof or a solvate thereof.

2. The compound as claimed in claim 1 wherein the compound has the following 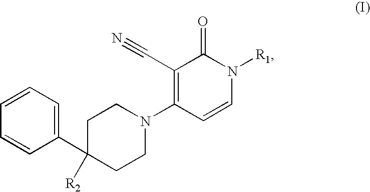
including any stereochemically isomeric form thereof, wherein
R₁ is C₄₋₆ alkyl, or C₁₋₃ alkyl substituted with C₃₋₇ cycloalkyl;
R₂ is hydrogen; fluoro; C₁₋₄ alkyl substituted with hydroxyl; C₁₋₄ alkyl substituted with fluoro; or C₁₋₄ alkyloxy substituted with fluoro;
or a pharmaceutically acceptable salt thereof or a solvate thereof.

3. The compound as claimed in claim 2 wherein the compound is other than

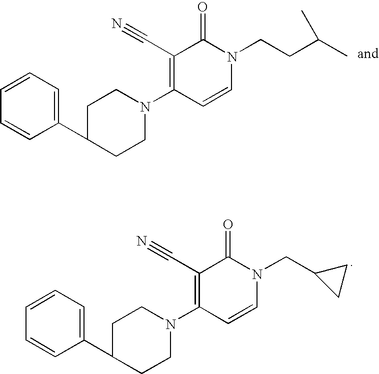

4. The compound as claimed in claim 2 wherein R₂ is fluoro; C_{1-4 alkyl substituted with hydroxyl; C}₁₋₄ alkyl substituted with fluoro; or C₁₋₄ alkyloxy substituted with fluoro.

5. The compound as claimed claim 1 wherein R₁ is C₄₋₆ alkyl.

6. The compound as claimed in claim 5 wherein R₁ is 1-butyl.

7. The compound as claimed in claim 1 wherein R₁ is C₁₋₃ alkyl substituted with C₃₋₇ cycloalkyl.

8. The compound as claimed in claim 7 wherein R₁ is cyclopropylmethyl.

9. The compound as claimed in claim 1, wherein R₂ is fluoro, hydrogen C₁₋₄ alkyl substituted with hydroxyl, C₁₋₄ alkyl substituted with fluoro, C₁₋₄ alkyloxy substituted with fluoro.

10. (canceled)

11. The compound as claimed in claim 1 wherein R₂ is methyl substituted with hydroxyl, methyl substituted with fluoro, or ethyloxy substituted with fluoro.

12-17. (canceled)

18. The compound as claimed in claim 1 wherein the compound has the following formula 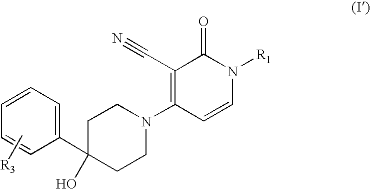
including any stereochemically isomeric form thereof, wherein
R₁ is C₄₋₆ alkyl, or C₁₋₃ alkyl substituted with C₃₋₇ cycloalkyl;
R₃ is hydrogen or halo;
or a pharmaceutically acceptable salt thereof or a solvate thereof.

19. The compound as claimed in claim 18 wherein R₃ is halo.

20. The compound as claimed in claim 18 wherein R₃ is chloro.

21. The compound as claimed in claim 20 wherein the compound is 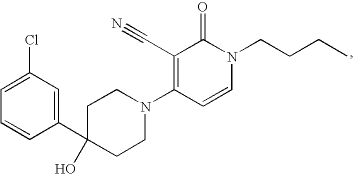
or a pharmaceutically acceptable salt thereof or a solvate thereof.

22. The compound as claimed in claim 1 wherein R₁ is 1-butyl, 3-methyl-1-butyl or cyclopropylmethyl; R₂ is hydrogen; fluoro; methyl substituted with hydroxyl; methyl substituted with fluoro; ethyloxy substituted with fluoro.

23. A compound as claimed in claim 1 wherein R₁ is 1-butyl or cyclopropylmethyl; R₂ is fluoro; methyl substituted with hydroxyl; methyl substituted with fluoro; ethyloxy substituted with fluoro.

24. The compound as claimed in claim 18 wherein R₁ is 1-butyl or cyclopropylmethyl; R₂ is hydrogen; fluoro or chloro.

25. A compound as claimed in claim 1 wherein the compound is selected from

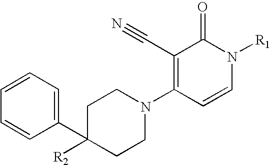

| 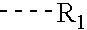 | 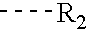 |
|---|---|
| 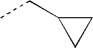 | 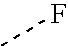 |
| 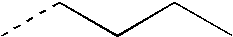 | 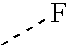 |
| 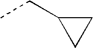 | 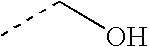 |
| 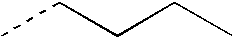 | 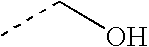 |
| 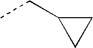 | 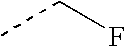 |
| 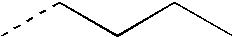 | 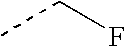 |
| 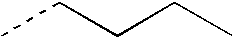 | 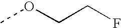 |
| 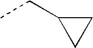 | H |
| 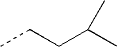 | H |

or a pharmaceutically acceptable salt thereof or a solvate thereof.

26. A compound as claimed in claim 1 wherein the compound is selected from

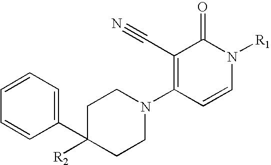

| 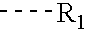 | 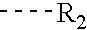 |
|---|---|
| 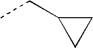 | 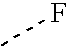 |
| 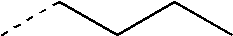 | 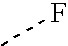 |
| 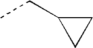 | 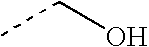 |
| 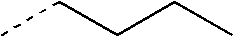 | 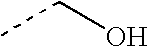 |
| 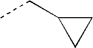 | 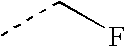 |
| 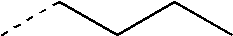 | 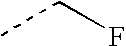 |
| 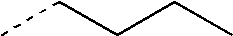 | 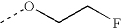 |

or a pharmaceutically acceptable salt thereof or a solvate thereof.

27. (canceled)

28. A pharmaceutical composition comprising a therapeutically
effective amount of a compound as claimed in claim 1 and a pharmaceutically acceptable carrier or diluent.

29. A compound as claimed in claim 1 for treating or preventing a condition in a mammal, including a human, the treatment or prevention of which is affected or facilitated by the neuromodulatory effect of a positive allosteric modulator of mGluR2.

30-45. (canceled)

46. A process for preparing a compound as claimed in claim 1, comprising
a) reacting an intermediate of formula (II) wherein Y represents a suitable leaving group, with an intermediate of formula (III) in a suitable reaction-inert solvent, in the presence of a suitable base, under heating conditions or reacting an intermediate of formula (II) with an intermediate of formula (III) in a suitable reaction-inert solvent, in the presence of a suitable base, a suitable catalyst, under heating conditions 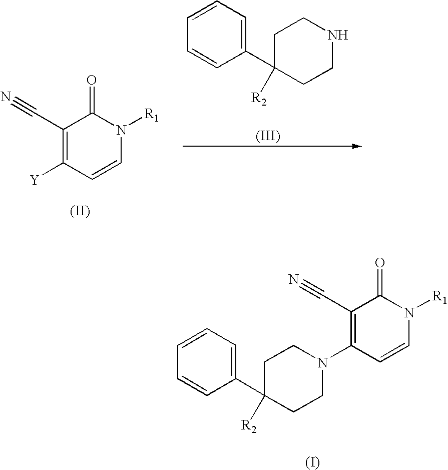
with R₁ and R₂ as defined in claim 1;
b) reacting a compound of formula (I-b) with a suitable fluorinating agent in a suitable reaction-inert solvent, under a moderately low temperature 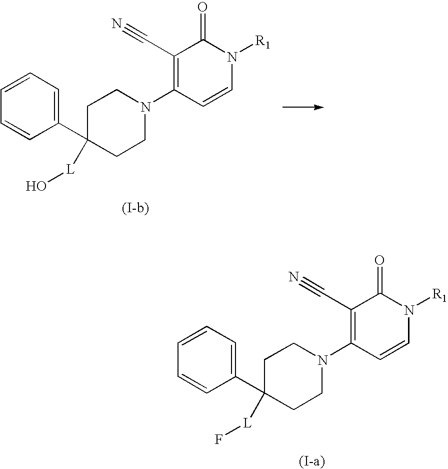
with L representing C₁₋₄alkyl and R₁ as defined in claim 1;
c) reacting an intermediate of formula (IV) with a suitable fluorinating agent in a suitable reaction-inert solvent, under a moderately low temperature 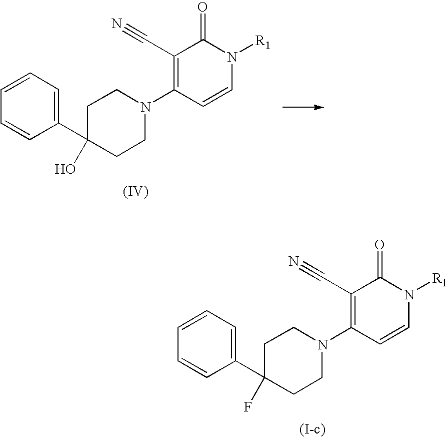
with R₁ as defined in claim 1;
d) hydrogenating an intermediate of formula (XIV) in a suitable solvent and in the presence of a suitable catalyst and a suitable base, 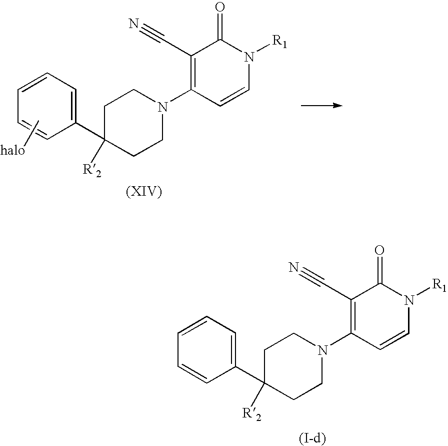
with R₁ as defined in claim 1 and R'2 representing C₁₋₄alkyloxy substituted with fluoro;
or, if desired, further converting compounds of formula (I) into each other following art-known transformations; or further, if desired, converting the compounds of formula (I), into a therapeutically active non-toxic acid addition salt by treatment with an acid, or conversely, converting the acid addition salt form into the free base by treatment with alkali; or, if desired, preparing stereochemically isomeric forms thereof.

47. A method for treating or preventing a condition in a mammal which is affected or facilitated by the neuromodulatory effect of a mGluR2 positive allosteric modulator, the method comprising administering to the mammal the compound of claim 1.

48. A method for treating or preventing a central nervous system disorder in a mammal, wherein the central nervous system disorder is selected from the group consisting of anxiety disorders, psychotic disorders, personality disorders, substance-related disorders, eating disorders, mood disorders, migraine, epilepsy or convulsive disorders, childhood disorders, cognitive disorders, neurodegeneration, neurotoxicity and ischemia, the method comprising administering to the mammal the compound of claim 1.

49. The method of claim 40, wherein the central nervous system disorder is (1) an anxiety disorder selected from the group consisting of agoraphobia, generalized anxiety disorder (GAD), obsessive-compulsive disorder (OCD), panic disorder, posttraumatic stress disorder (PTSD), social phobia and other phobias; (2) a psychotic disorder selected from the group consisting of schizophrenia, delusional disorder, schizoaffective disorder, schizophreniform disorder and substance-induced psychotic disorder; (3) a personality disorder selected from the group consisting of obsessive-compulsive personality disorder and schizoid, schizotypal disorder; (4) a substance-related disorder selected from the group consisting of alcohol abuse, alcohol dependence, alcohol withdrawal, alcohol withdrawal delirium, alcohol-induced psychotic disorder, amphetamine dependence, amphetamine withdrawal, cocaine dependence, cocaine withdrawal, nicotine dependence, nicotine withdrawal, opioid dependence and opioid withdrawal; (5) an eating disorder selected from the group consisting of anorexia nervosa and bulimia nervosa; (6) a mood disorder selected from the group consisting of bipolar disorders (I & II), cyclothymic disorder, depression, dysthymic disorder, major depressive disorder and substance-induced mood disorder; (7) a migraine; (8) epilepsy or a convulsive disorder selected from the group consisting of generalized nonconvulsive epilepsy, generalized convulsive epilepsy, petit mal status epilepticus, grand mal status epilepticus, partial epilepsy with or without impairment of consciousness, infantile spasms, epilepsy partialis continua, and other forms of epilepsy; (9) a childhood disorder selected from the group consisting of attention-deficit/hyperactivity disorder; and (10) a cognitive disorder selected from the group consisting of delirium, substance-induced persisting delirium, dementia, dementia due to HIV disease, dementia due to Huntington's disease, dementia due to Parkinson's disease, dementia of the Alzheimer's type, substance-induced persisting dementia and mild cognitive impairment.

50. The method of claim 47, wherein the method further comprises administering to the mammal an orthosteric agonist of mGluR2 in combination with the compound of claim 1.

51. The method of claim 48, wherein the method further comprises administering to the mammal an orthosteric agonist of mGluR2 in combination with the compound of claim 1.
